(19) United States
(12) Patent Application Publication
(10) Pub. No.: US 2023/0147669 A1
(43) Pub. Date: May 11, 2023

(54) SCREWLESS INTERBODY DEVICE FOR SPINAL SURGERY

(71) Applicant: RV MEDICAL LLC, Evergreen, CO (US)

(72) Inventors: David VanSickle, Denver, CO (US); Ryan Rooney, Evergreen, CO (US)

(21) Appl. No.: 17/918,473

(22) PCT Filed: Apr. 22, 2021
(86) PCT No.: PCT/US2021/028622

§ 371 Date: Oct. 12, 2022

Related U.S. Application Data
(60) Provisional application No. 63/014,542, filed on Apr. 23, 2020.

(51) Int. Cl. A61F 2/44

(52) U.S. Cl. CPC A61F 2/4455; A61F 2002/30535

(57) ABSTRACT

The present disclosure is generally directed to an interbody device for use in a spinal fusion procedure. The interbody device generally includes a superior endplate opposite to an inferior endplate. Friction elements extend from at least one of the superior endplate and the inferior endplate. In some embodiments, one or more of the friction elements comprise a plurality of teeth. In embodiments, the teeth of one or more of the friction elements are arranged in a row.

### CROSS REFERENCE TO RELATED APPLICATIONS

This application claims priority and benefits under 35 U.S.C. § 119(e) to U.S. Provisional Patent Application Ser. No. 63/014,542 filed on Apr. 23, 2020, which is incorporated herein in its entirety by reference.

### FIELD

The current disclosure is generally directed to interbody devices inserted between adjacent vertebrae to stabilize the intervertebral space. The interbody devices also facilitate fusion of adjacent vertebrae. Friction elements extend from a superior endplate and an inferior endplate of the interbody devices to engage the adjacent vertebrae to prevent unintended or inadvertent movement of the interbody devices.

### SUMMARY

Anterior cervical discectomy and fusions (ACDF) were developed in the 1950's and are a common surgery performed by neurosurgeons and orthopedic spine surgeons. The surgeries are utilized to alleviate compression of the spinal cord or the exiting nerve roots which can cause myelopathy and radiculopathy respectively. The surgeries are also utilized for cases where the cervical spine is unstable due to ligamentous injury, fracture, or degenerative disease.

A similar surgery called an anterior lumbar instrumentation and fusion (ALIF) is used for similar conditions in a lumbar spine. The main differences in the lumbar spine are that the spinal elements are much larger requiring larger instrumentation and larger implants. Another key difference is that the spinal cord is typically not present in the lumbar spine ending at approximately the L1 level and therefore myelopathy is not typically an issue with the lumbar spine.

Current surgical technique calls for the removal of disc material from an anterior approach either through the neck or through the abdomen. This portion of the surgery alleviates the compression but creates instability and would allow for one vertebral body to collapse on another if no spacer were to be placed between the vertebral bodies.

Initially, when these techniques were developed, a bone graft was used as a spacer. The bone graft could be either autologous (harvested from the patient) or from a bone donor.

As the ACDF and ALIF techniques were further developed, surgeons began fixing a plate over the surface of the bone implant with anchoring screws in the vertebral body above and the vertebral body below. This is the most common technique today. However, harvesting autologous bone graft from the patient is painful and use of donor graft material is expensive. Additionally, accurately orienting the screws for the plate is difficult and time consuming, increasing the risk and time required to perform the ACDF or ALIF surgery.

Later, and referring now to FIG. 1, artificial cages 12A came into use to replace the bone graft used in ACDF and ALIF surgeries. The cages can be manufactured from either plastic or metal with the typical plastic cage consisting of PEEK and the typical metal cage consisting of titanium. The cages are frequently designed to allow either autologous or donor bone to be packed within them to allow for boney fusion to occur through the spacer. Although the cage eliminates the need to harvest bone from the patient, after the artificial cage is inserted into the disc space 6 between adjacent vertebrae 2, a plate 8 is still used and is fixed to the adjacent vertebrae. Thus, ACDF and ALIF surgeries performed with cages still suffer from the problems and inefficiencies required to properly orient and insert screws 10 into the vertebral body 2A above and the vertebral body 2B below the cage.

More recently, further development of the ACDF and ALIF techniques resulted in the introduction of a stand-alone cage that does not require the use of a plate. The cage is anchored to one or more of the adjacent vertebrae by screws 14. Examples of cages 12B for ACDF surgeries are illustrated in FIG. 2 and cages 12C used in ALIF surgery are shown in FIG. 3. In many cases the cages 12B, 12C are fixated to the upper and lower vertebral bodies in exactly the same way as a plate. The cages use two or more screws 14 oriented at an angle to the cage to fix the cage to the adjacent vertebral bodies. At least one screw 14 is anchored into the vertebral body cranially (or upwardly) and at least one screw anchored into the vertebral body caudally (or downwardly). Alternative fixation devices other than screws are less commonly used, such as blades or shims 16 (shown in FIGS. 2E-2G) which can be projected into the vertebral body cranially and caudally.

Typically placing the screws 14 or shims 16 of the cage 12B, 12C is the most difficult and time consuming portion of the ACDF and ALIF procedures due to the angle which must be achieved to set the screws or shims. It is difficult to orient the instruments used to set the screws or shims at the required angle within the surgical space. Additionally, the instruments needed to place the screws create a risk of injury to critical structures in the neck or abdomen of the patient. Moreover, if a screw or shim is not properly oriented, or if a screw is over tightened, the vertebral body may be damaged. Further, apertures formed in the vertebrae for the screws can weaken the vertebrae, especially when the vertebrae is already damaged or weakened due to disease.

All of the cages 12A, 12B, and 12C of the related art shown in FIGS. 1-3 require some form of fixation. The fixation may be a plate to hold the cage within the disc space or screws to fix the cage to the upper and lower vertebrae. Without the fixation, the cages will move unintentionally resulting in unsuccessful fusion of the vertebrae.

An alternative to spinal fusion provided by ACDF and ALIF procedures is a disc replacement procedure. Referring now to FIG. 4, disc replacement devices 18 are shown. The disc replacement device is an alternative to a spacer made from bone and to a cage 12. The disc replacement devices are configured to move and articulate to preserve some motion between adjacent vertebrae. Accordingly, disc replacement devices are not suitable for use in a spinal fusion procedure, such as ACDF and ALIF procedures.

Disc replacement devices 18 typically include a superior or upper endplate 20A flexibly joined to an inferior or lower endplate 20B by a medial element 22. The medial element 22 may comprise a plastic or a disc of polyethylene. The cranial (upper) and caudal (lower) endplates may include micro porous areas to allow for bone to grow.

Some disc replacement devices 18 are designed to be fixated initially with simple friction. The endplates 20A, 20B may include a keel 24 or teeth 26 to engage one or more of the adjacent vertebral bodies. These friction fit disc replacement devices 18 are noted to be easy to place because no anchor screws are required. Disc replacement devices 18 have shown good fixation without significant complications due to migration out of the intended location.

Currently there is no cage available for use in ACDF and ALIF procedures which combines the ease of a friction fit device with a simple spacer which is not designed to act as a disc replacement. Accordingly, there is an unmet need for an interbody device for use in fusion procedures that frictionally engages an upper and a lower vertebral body.

It is one aspect of the present disclosure to provide an interbody device to frictionally engage an upper vertebral body and a lower vertebral body and which facilitates fusion of the upper and lower vertebral bodies.

In embodiments, a superior endplate of the interbody device includes a friction element.

In embodiments, an inferior endplate of the interbody device includes a friction element.

In embodiments, the friction element extends at least a portion of a length of an endplate between an anterior surface and a posterior surface of the interbody device.

Optionally, the friction element is oriented approximately parallel to a median plane of the interbody device. Alternatively, in another embodiment, a friction element is oriented at an oblique angle to the median plane.

In some embodiments, the friction element comprises a keel projecting from an endplate of the interbody device.

In another embodiment, the friction element comprises a tooth projecting from an endplate of the interbody device.

Optionally, the friction element comprises a plurality of teeth arranged in a row.

In embodiments, the teeth have free ends that are pointed.

In embodiments, free ends of at least some of the teeth are oriented toward an anterior surface of the interbody device.

Optionally, for any of the embodiments, a free end of at least one tooth is oriented toward a posterior surface of the interbody device.

Optionally, the superior endplate includes two rows of teeth.

In embodiments, a first row of teeth of the superior endplate extends along a first axis and the second row of teeth of the superior endplate extends along a second axis. In some embodiments, the first axis is approximately parallel to the second axis. Alternatively, in another embodiment, the first axis is oriented at an oblique angle to the second axis.

Optionally, the inferior endplate includes two rows of teeth.

In some embodiments, a third row of teeth of the inferior endplate extends along a third axis and the fourth row of teeth of the inferior endplate extends along a fourth axis. In embodiments, the third axis is approximately parallel to the fourth axis. Alternatively, in another embodiment, the first axis is oriented at an oblique angle to the second axis.

In some embodiments, the first axis is oriented at an oblique angle to the third axis.

In embodiments, the superior endplate includes a friction element that is offset from a friction element of the inferior endplate. Optionally, the superior friction element extends along a first axis and the inferior friction elements extends along a second axis. In some embodiments, the first axis is approximately parallel to the second axis. Alternatively, in another embodiment, the first axis is oriented at an oblique angle to the second axis.

Optionally, in any of the embodiments, the superior endplate may be generally planar.

Alternatively, in another embodiment, the superior endplate includes a dome the projects upwardly (or cranially).

Optionally, in any of the embodiments, the inferior endplate may be generally planar.

Alternatively, in another embodiment, the inferior endplate includes a dome the projects downwardly (or caudally).

In embodiments, one or more of the superior endplate and the inferior endplate comprise a micro porous material for bone ingrowth and/or adherence.

In some embodiments, the interbody device comprises a plastic.

In embodiments, the plastic is a thermoplastic polymer.

Optionally, the plastic is a polyether ether ketone (PEEK).

Additionally, or alternatively, in other embodiments, the interbody device comprises a metal.

In some embodiments, the metal is titanium or a titanium alloy.

In embodiments, the interbody device is produced by an additive manufacturing process.

Optionally, for any of the embodiments, the interbody device may be produced by a 3-D printing process.

In embodiments, the interbody device is treated to produce micro- and nanoscale surface roughness.

In some embodiments, the treatment of the interbody device includes acid etching to promote the micro- and nanoscale surface roughness.

Additionally, or alternatively, the interbody device is heat treated to promote the micro- and nanoscale surface roughness.

Optionally, for any of the embodiments, the interbody device may be rigid.

In embodiments, the interbody device is of a one-piece construction.

One aspect of the present disclosure is an interbody device for a spinal fusion procedure which frictionally engages an upper vertebral body and a lower vertebral body. The interbody device comprises: (1) a superior endplate opposite to an inferior endplate; (2) a superior friction element extending from the superior endplate; (3) an inferior friction element extending from the inferior endplate; and (4) an anterior surface including an engagement feature.

In some embodiments, the superior friction element extends generally linearly. The friction element may extend approximately parallel to a median plane bisecting the interbody device. Alternatively, the friction element is oriented at an oblique angle to the median plane.

Optionally, two superior friction elements extend from the superior endplate. In some embodiments, at least one inferior friction element extends from the inferior endplate.

In some embodiments, a first one of the superior friction elements extends along a first axis and a second one of the superior friction elements extends along a second axis. Optionally, the first axis is approximately parallel to the second axis. Alternatively, in another embodiment, the first axis is oriented at an oblique angle to the second axis.

Optionally, an inferior friction element extends along a third axis. In some embodiments, the third axis is oriented at an oblique angle to at least one of the first axis and the second axis.

Additionally, the inferior friction element can optionally be offset from the superior friction elements. In embodiments, the inferior friction element is offset medially from one or more of the superior friction elements.

In some embodiments, the friction elements comprise a keel extending from the endplates.

Optionally, the keel includes projections extending from lateral surfaces of the keel.

In some embodiments, the projections are movable to a first position to facilitate movement of the interbody device in a posterior direction.

Additionally, or alternatively, the projections can be movable to a second position to limit movement of the interbody device in an anterior direction.

For any of the embodiments, the friction elements may optionally comprise a plurality of teeth arranged in a row.

Optionally, the teeth include a free end that is pointed.

Additionally, or alternatively, one or more of the endplates of the interbody device can include a dome.

In embodiments, one or more of the superior endplate and the inferior endplate comprise a micro porous material for bone ingrowth.

Optionally, the interbody device comprises a PEEK.

In embodiments, the interbody device comprises a metal.

In some embodiments, the interbody device is rigid.

In embodiments, the interbody device is produced by an additive manufacturing process.

In embodiments, the interbody device is configured to frictionally engage the upper vertebral body and the lower vertebral body without the use of a screw and without the use of a plate.

It is another aspect of the present disclosure to provide a method of performing a spinal fusion procedure. The method comprises: (1) removing disc material from a disc space between an upper vertebral body and a lower vertebral body; (2) positioning an interbody device in the disc space, the interbody device generally including: (a) a superior endplate with a superior friction element to engage the upper vertebral body; and (b) an inferior endplate opposite to the superior endplate, the inferior endplate including an inferior friction element to engage the lower vertebral body. The interbody device is retained in a predetermined position within the disc space by frictional engagement of the friction elements with the vertebral bodies.

In embodiments, no screws or plates are used to retain the interbody device in the predetermined position. Optionally, the interbody device has no moving parts.

For any of the embodiments, the friction elements optionally comprise one of a keel and a plurality of teeth arranged in one or more rows.

Optionally, the method further comprises forming a slot in the upper vertebral body to receive the superior friction element.

In some embodiments, positioning the interbody device in the disc space includes aligning a friction element extending from the superior endplate with the slot in the upper vertebral body.

Additionally, or alternatively, the method can include forming a slot in the lower vertebral body to receive the inferior friction element.

In embodiments, the slot in the upper vertebral body is formed such that it is positioned offset medially from the slot in the lower vertebral body.

Optionally, the slot in one or more of the upper vertebral body and the lower vertebral body is formed by a cutting instrument.

In some embodiments, positioning the interbody device in the disc space includes aligning a friction element extending from the inferior endplate with the slot in the lower vertebral body.

Optionally, the method includes placing bone graft material in the disc space.

In any of the embodiments, the method optionally includes packing bone graft material in the interbody device.

Alternatively, in another embodiment, no bone graft or bone graft substitute is positioned within the disc space with the interbody device.

In embodiments, no screws are positioned in the upper vertebral body or the lower vertebral body during the spinal fusion procedure.

In some embodiments, the interbody device includes a dome extending from one or more of the superior endplate and the inferior endplate.

In embodiments, positioning the interbody device in the disc space includes aligning a dome extending from the superior endplate with a recess in the upper vertebral body.

Additionally, or alternatively, positioning the interbody device in the disc space may include aligning a dome extending from the inferior endplate with a recess in the lower vertebral body.

Another aspect of the present disclosure is a method of forming an interbody device for a spinal fusion procedure which is configured to engage one or more of an upper vertebral body and a lower vertebral body. The method comprises: (1) forming the interbody device with a superior endplate opposite to an inferior endplate; (2) forming a superior friction element extending from the superior endplate; and (3) forming an inferior friction element extending from the inferior endplate.

Optionally, the method can include forming an engagement feature on an anterior surface of the interbody device.

In some embodiments, the friction elements include one or more of a bump, a ridge, a tooth, and a projection.

In embodiments, the superior friction is formed to extend generally alone an axis. In some embodiments, the axis extends approximately parallel to a median plane bisecting the interbody device. Alternatively, the axis extends at an oblique angle to the median plane.

Optionally, forming the friction elements comprises forming two superior friction elements extending from the superior endplate and forming at least one inferior friction element that extends from the inferior endplate.

In embodiments, forming the superior and inferior friction elements comprises offsetting the inferior friction element medially from the superior friction elements. In this manner, the superior and inferior friction elements are not co-planar.

In some embodiments, forming the friction elements comprises forming a keel that extends from the endplates.

Optionally, the keel is formed with projections extending from lateral surfaces of the keel.

In embodiments, the projections are movable to a first position to facilitate movement of the interbody device in a posterior direction.

Additionally, or alternatively, the projections are formed to be movable to a second position to limit movement of the interbody device in an anterior direction.

For any of the embodiments, forming the friction elements optionally comprises forming a plurality of teeth arranged in a row.

Optionally, the teeth are formed with a free end that is pointed.

Additionally, or alternatively, the method includes forming a dome that projects from one or more of the endplates of the interbody device.

The method can also include forming a micro porous structure for bone ingrowth in one or more of the superior endplate and the inferior endplate.

In some embodiments, the interbody device comprises a plastic.

In embodiments, the plastic is a thermoplastic polymer.

Optionally, the interbody device comprises a PEEK.

In embodiments, the interbody device comprises a metal.

In some embodiments, the metal is titanium or a titanium alloy.

In any of the embodiments, the interbody device optionally is rigid.

Optionally, the method includes casting at least a portion of the interbody device.

Additionally, or alternatively, the method includes forging a portion of the interbody device.

In embodiments, the method includes forming, or finishing, a portion of the interbody device by an additive manufacturing process.

Optionally, the method includes forming micro- or nano-scale roughness by a 3-D additive process on a portion of the interbody device that has been cast and/or forged.

For any of the embodiments, the interbody device is optionally produced by a 3-D printing process.

In embodiments, the interbody device is of a one-piece construction. For example, the interbody device may be formed to have no moving parts.

In embodiments, the interbody device is configured to frictionally engage the upper vertebral body and the lower vertebral body without the use of a screw and without the use of a plate.

Optionally, the interbody device is formed with an interior cavity.

In embodiments, the method includes treating the interbody device to produce micro- and nanoscale surface roughness.

In some embodiments, the treatment of the interbody device includes acid etching to promote the micro- and nanoscale surface roughness.

Additionally, or alternatively, the method includes heat treating the interbody device to promote the micro- and nanoscale surface roughness.

One aspect of the present disclosure is an interbody device as substantially described herein.

Another aspect of the present disclosure is a method of using an interbody device as substantially described herein in a surgical procedure.

Still another aspect of the present disclosure is a method of forming an interbody device as substantially described herein.

Another aspect of the present disclosure is an interbody device for use in a spinal fusion procedure to frictionally engage an upper vertebral body and a lower vertebral body, comprising: (1) a superior endplate opposite to an inferior endplate; (2) a first lateral wall opposite to a second lateral wall; (3) an anterior wall opposite to a posterior wall, the anterior wall including an engagement feature; (4) a first friction means extending from the superior endplate and oriented at an oblique angle to a median plane that extends through the superior and inferior endplates and bisects the interbody device; and (5) a second friction means extending from the inferior endplate.

In embodiments, the friction means extend along a predetermined length.

In embodiments, the friction means have a predetermined height.

In embodiments, the friction means are keels.

In embodiments, the friction means each comprise a plurality of teeth that extend in a row.

One aspect of the present disclosure is an interbody device configured for use in a spinal fusion procedure to frictionally engage an upper vertebral body and a lower vertebral body, comprising: (1) a superior endplate opposite to an inferior endplate; (2) a first superior friction element extending from the superior endplate; (3) a first inferior friction element extending from the inferior endplate; and (4) an anterior wall including an engagement feature.

In embodiments, the first superior friction element is oriented at an oblique angle to a median plane that extends through the superior and inferior endplates and bisects the interbody device.

In embodiments, the interbody device further comprises a second superior friction element extending from the superior endplate.

In embodiments, the first superior friction element is on a first side of the median plane and the second superior friction element is on a second side of the median plane.

In embodiments, the second superior friction element is oriented at an oblique angle to the median plane and to the first superior friction element.

In embodiments, the first superior friction element is oriented at a first angle relative to the median plane and the second superior friction element is oriented at a second angle relative to the median plane.

In embodiments, the second angle is a positive angle.

In embodiments, the first angle is a negative angle that is of approximately equal magnitude to the second angle.

In embodiments, the interbody device further comprises a third superior friction element extending from the superior endplate.

In embodiments, the interbody device includes one or more of the previous embodiments and further comprises a second inferior friction element extending from the inferior endplate.

In embodiments, the second inferior friction element is on the first side of the median plane and the first inferior friction element is on the second side of the median plane.

In embodiments, the first inferior friction element is oriented at a third angle relative to the median plane.

In embodiments, the third angle is approximately equal to the first angle such that the first inferior friction element is approximately parallel to the first superior friction element.

In embodiments, the second inferior friction element is oriented at a fourth angle relative to the median plane.

In embodiments, the fourth angle is approximately equal to the second angle such that the second inferior friction element is approximately parallel to the second superior friction element.

In embodiments, the interbody device further comprises a third inferior friction element extending from the inferior endplate.

In embodiments, the interbody device includes one or more of the previous embodiments and one or more of the friction elements each comprise a plurality of teeth.

In embodiments, the plurality of teeth are arranged in a row.

In embodiments, the row of teeth is substantially linear.

In embodiment, the row of teeth extends along an arcuate path.

Alternatively, in embodiments, the plurality of teeth are not arranged in a row.

In embodiments, the teeth include: (a) a first side extending away from one of the superior and inferior endplates, the first side being proximate to the anterior wall; (b) a long side that is oriented at an oblique angle to the first side; and (c) a free end defined by an intersection of the long side with the first side.

In embodiments, the first side is approximately planar and the long side is approximately planar.

In embodiments, the interbody device includes one or more of the previous embodiments and further comprises a posterior wall positioned opposite to the anterior wall.

In embodiments, the posterior wall has a first height that is less than a second height of the anterior wall.

In embodiments, the superior endplate extends along a curved path between the anterior wall and the posterior wall such that the superior endplate is convex between the anterior and superior walls.

Additionally, or alternatively, in embodiments, the inferior endplate extends along a curved path between the anterior wall and the posterior wall such that the inferior endplate is convex between the anterior and superior walls.

In embodiments, the interbody device includes one or more of the previous embodiments and one or more of the superior endplate and the inferior endplate comprise a micro porous material for bone ingrowth.

In embodiments, the interbody device comprises a PEEK and includes one or more of the previous embodiments.

In embodiments, the interbody device comprises a metal and includes one or more of the previous embodiments.

In embodiments, the interbody device is rigid and includes one or more of the previous embodiments.

In embodiments, the interbody device is produced by an additive manufacturing process and includes one or more of the previous embodiments.

In embodiments, the interbody device includes one or more of the previous embodiments and the interbody device is configured to frictionally engage the upper vertebral body and the lower vertebral body without the use of a secondary anchor, such as a screw, a pin, a plate, and a rod.

In embodiments, the interbody device does not include any movable parts, such as deployable anchors or projections.

In embodiments, the interbody device includes one or more of the previous embodiments and the interbody device is static.

In embodiments, the interbody device includes one or more of the previous embodiments and further comprises one or more of: (a) a first lateral plate opposite to a second lateral plate; and (b) an interior cavity within the interbody device.

In embodiments, an aperture formed through one or more of the first and second lateral plates extends to the interior cavity.

In embodiments, the interbody device includes one or more of the previous embodiments and further comprises an interior cavity within the interbody device.

In embodiments, an orifice formed through one or more of the superior endplate and the inferior endplate extends to the interior cavity.

In embodiments, the interbody device includes one or more of the previous embodiments and the engagement feature includes an aperture that extends to an interior cavity within the interbody device.

Yet another aspect of the present disclosure is a method of performing a spinal fusion procedure, comprising: (1) removing disc material from a disc space between an upper vertebral body and a lower vertebral body; and (2) positioning an interbody device according to the present disclosure in the disc space such that a first superior friction element of the interbody device is oriented to engage the upper vertebral body, and a first inferior friction element is oriented to engage the lower vertebral body. In this manner, the interbody device is retained in a predetermined position within the disc space by frictional engagement of the friction elements with the vertebral bodies.

In embodiments, the interbody device includes: (a) a superior endplate opposite to an inferior endplate; (b) the first superior friction element extending from the superior endplate; (c) the first inferior friction element extending from the inferior endplate; and (d) an anterior wall including an engagement feature.

In embodiments, no screws or plates are used to retain the interbody device in the predetermined position.

In embodiments, the method further comprises one or more of: (a) forming a slot in the upper vertebral body to receive the superior friction element; and (b) forming a slot in the lower vertebral body to receive the inferior friction element.

In embodiments, the friction elements comprise one of a keel and a plurality of teeth.

In embodiments, the plurality of teeth are arranged in one or more rows.

In embodiment, the interbody device frictionally engages the upper vertebral body and the lower vertebral body without the use of a secondary anchor, such as a screw, a pin, a plate, and a rod.

It is another aspect of the present disclosure to provide a method of forming an interbody device configured for use in a spinal fusion procedure which is configured to engage one or more of an upper vertebral body and a lower vertebral body, comprising: (1) forming the interbody device with a superior endplate opposite to an inferior endplate; (2) forming a first friction element extending from the superior endplate; (3) forming a second friction element extending from the superior endplate; (4) forming a third friction element extending from the inferior endplate; (5) forming a fourth friction element extending from the inferior endplate; (6) forming an anterior wall extending between the superior and inferior endplates; and (7) forming an engagement feature that includes an aperture extending through the anterior wall to an interior cavity within the interbody device.

In embodiments, the first friction element extends at a first oblique angle to a median plane extending through the endplates and bisecting the interbody device.

In embodiments, the second friction element extends at a second oblique angle to the median plane.

In embodiments, the third friction element extends at a third oblique angle to the median plane.

In embodiments, the third friction element is approximately parallel to the first friction element.

In embodiments, the fourth friction element extends at a fourth oblique angle to the median plane.

In embodiments, the fourth friction element is approximately parallel to the second friction element.

In embodiments, the method includes one or more of the previous embodiments and further comprises forming a fifth friction element extending from one of the superior and inferior endplates.

In embodiments, the method includes one or more of the previous embodiments and further comprises forming a sixth friction element extending from one of the superior and inferior endplates.

In embodiments, one or more of the friction elements each comprise a plurality of teeth.

In embodiments, the teeth of the friction elements are arranged in a row.

Alternatively, in embodiment, the teeth of at least one of the friction elements are not arranged in a row.

In embodiments, the method includes forming the interbody device by an additive manufacturing process and the interbody devices includes one or more of the previous embodiments.

In embodiments, the method includes one or more of the previous embodiments and the interbody device is static.

In embodiments, the interbody device is formed of one or more of a PEEK and a metal.

In embodiments, the method includes one or more of the previous embodiments and further comprises treating the interbody device to produce micro- or nanoscale surface roughness.

In embodiments, the method includes acid etching the interbody device to promote the micro- or nanoscale surface roughness.

In embodiments, the method includes one or more of the previous embodiments and further comprises heat treating the interbody device to promote micro- or nanoscale surface roughness.

In embodiments, the method includes one or more of the previous embodiments and one or more of the superior endplate and the inferior endplate comprise a micro porous material for bone ingrowth.

In embodiments, the method includes one or more of the previous embodiments and further comprises finishing one or more exterior surface of the interbody device by shot blasting.

The Summary is neither intended nor should it be construed as being representative of the full extent and scope of the present disclosure. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more clear from the Detailed Description, particularly when taken together with the drawings.

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

The term “a” or “an” entity, as used herein, refers to one or more of that entity. As such, the terms “a” (or “an”), “one or more” and “at least one” can be used interchangeably herein.

Unless otherwise indicated, all numbers expressing quantities, dimensions, conditions, ratios, ranges, and so forth used in the specification and claims are to be understood as being modified in all instances by the term “about” or “approximately”. Accordingly, unless otherwise indicated, all numbers expressing quantities, dimensions, conditions, ratios, ranges, angles, relationships and so forth used in the specification and claims may be increased or decreased by approximately 10% to achieve satisfactory results. Additionally, where the meaning of the terms “about” or “approximately” as used herein would not otherwise be apparent to one of ordinary skill in the art, the terms “about” and “approximately” should be interpreted as meaning within plus or minus 10% of the stated value.

The term “parallel” means two objects are oriented at an angle within plus or minus 0° to 5° unless otherwise indicated. Similarly, the term “perpendicular” means two objects are oriented at angle of from 85° to 95° unless otherwise indicated.

All ranges described herein may be reduced to any sub-range or portion of the range, or to any value within the range without deviating from the invention. For example, the range “5 to 55” includes, but is not limited to, the sub-ranges “5 to 20” as well as “17 to 54.”

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C., Section 112(f). Accordingly, a claim incorporating the term “means” shall cover all structures, materials, or acts set forth herein, and all of the equivalents thereof. Further, the structures, materials, or acts and the equivalents thereof shall include all those described in the Summary, Brief Description of the Drawings, Detailed Description, Abstract, and Claims themselves.

### BRIEF DESCRIPTION OF THE DRAWINGS

The accompanying drawings, which are incorporated in and constitute a part of the specification, illustrate embodiments of the disclosed system and together with the general description of the disclosure given above and the detailed description of the drawings given below, serve to explain the principles of the disclosed system(s) and device(s).

FIG. 1 illustrates a plate fixed to vertebrae of a patient;

FIGS. 2A, 2B, 2C and 2D are views of cages related to the present disclosure and which are used in ACDF procedures and which include screws to anchor the cages;

FIGS. 2E, 2F and 2G are views of cages for ACDF procedures that have shims to anchor the cages;

FIGS. 3A, 3B, and 3C show several views of a cage related to the present disclosure with screws and which is used in ALIF procedures;

FIGS. 4A, 4B, and 4C are illustrations of a disc replacement device related to the present disclosure and which includes a keel to anchor the device;

FIGS. 4D, 4E, 4F and 4G show another disc replacement device of the present disclosure that is anchored by teeth that project from the upper and lower endplates;

FIGS. 5A, 5B, 5C and 5D illustrate an interbody device of one embodiment of the present disclosure;

FIGS. 6A, 6B, 6C, 6D and 6E illustrate another interbody device according to embodiments of the present disclosure and which includes a keel to anchor the interbody device to adjacent vertebrae;

FIG. 6F is an anterior (or front) elevation schematic view of interbody devices of the present disclosure positioned within disc spaces between vertebrae;

FIGS. 7A, 7B, 7C and 7D are views of still another interbody device according to embodiments of the present disclosure and which includes teeth to anchor the interbody device to adjacent vertebrae;

FIGS. 8A, 8B, and 8C illustrate embodiments of interbody devices of the present disclosure which include one or more open endplates or open front/rear plates;

FIGS. 9A and 9B illustrate additional embodiments of interbody devices with open endplates and/or open front/rear plates;

FIG. 10 is a front elevation view of an interbody device similar to the interbody device of FIG. 6 and which includes an upper endplate with a dome;

FIG. 11 is another front elevation view of an interbody device of the present disclosure which includes domes on the upper and lower endplates;

FIG. 12 is a front elevation view of an interbody device similar to the interbody device of FIG. 7 and which includes an upper endplate with a dome;

FIG. 13 is another front elevation view of an interbody device of the present disclosure which includes domes on the upper and lower endplates;

FIGS. 14 and 15 are perspective views of an interbody device according to another embodiment of the present disclosure;

FIG. 16 is a top plan view of the interbody device of FIG. 14;

FIG. 17 is a bottom plan view of the interbody device of FIG. 14;

FIG. 18 is a front elevation view of the interbody device of FIG. 14;

FIG. 19 is a rear elevation view of the interbody device of FIG. 14;

FIG. 20 is a side elevation view of a first lateral side of the interbody device of FIG. 14, the second lateral side being substantially the same;

FIG. 21 is a cross-sectional side elevation view of the interbody device of FIG. 14, the cross-section taken along a median plane that bisects the interbody device;

FIG. 22 is a cross-sectional perspective view of the interbody device of FIG. 14 taken along a transverse plane that bisects the interbody device;

FIG. 23 is a perspective view of interbody devices according to the embodiment of FIG. 14, the interbody devices being of different sizes;

FIG. 24A is a graph of the performance of samples of an interbody device of the present disclosure in a static expulsion test conducted with simulated bone of Grade 5;

FIG. 24B is a graph of the performance of three samples of an AIS-C Cervical Stand-Alone System produced by Genesys Spine in the static expulsion test conducted with simulated bone of Grade 5;

FIG. 25A is a graph of the performance of three samples of the interbody device of the present disclosure in the static expulsion test conducted with simulated bone of Grade 15;

FIG. 25B is a graph of the performance of three samples of the AIS-C Cervical Stand-Alone System produced by Genesys Spine in the static expulsion test conducted with simulated bone of Grade 15;

FIG. 26A is a graph of the performance of three samples of the interbody device of the present disclosure in the static expulsion test conducted with simulated bone of Grade 40; and

FIG. 26B is a graph of the performance of three samples of the AIS-C Cervical Stand-Alone System produced by Genesys Spine in the static expulsion test conducted with simulated bone of Grade 40.

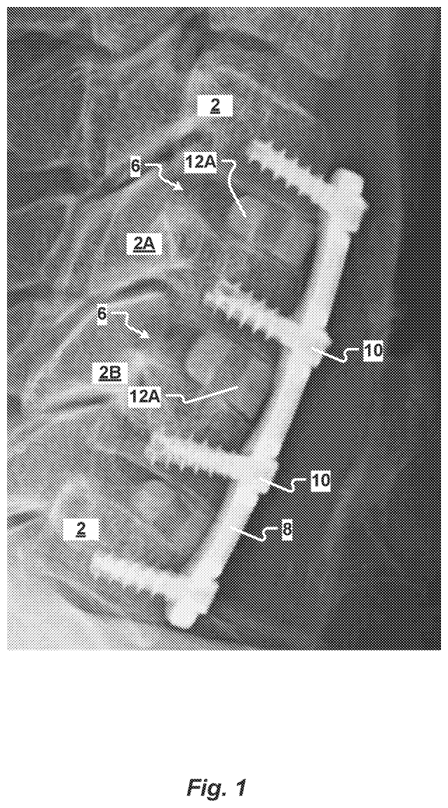

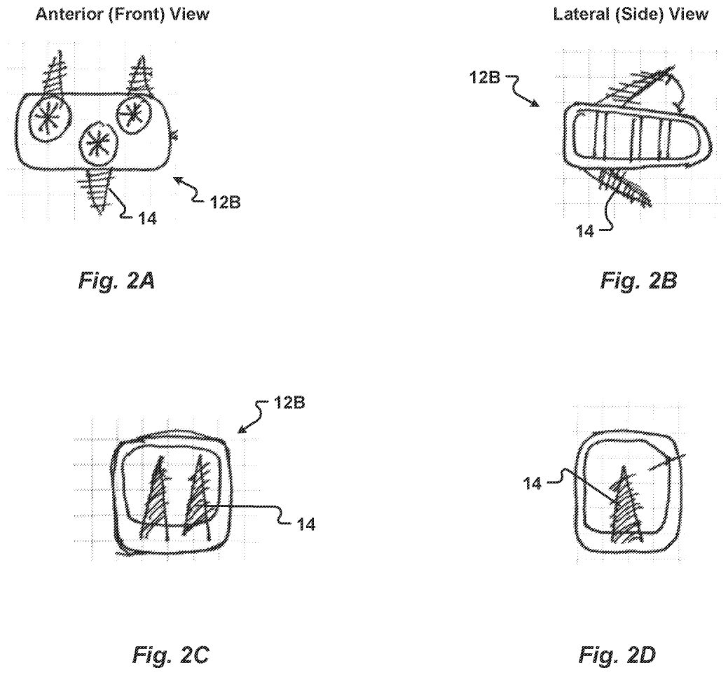

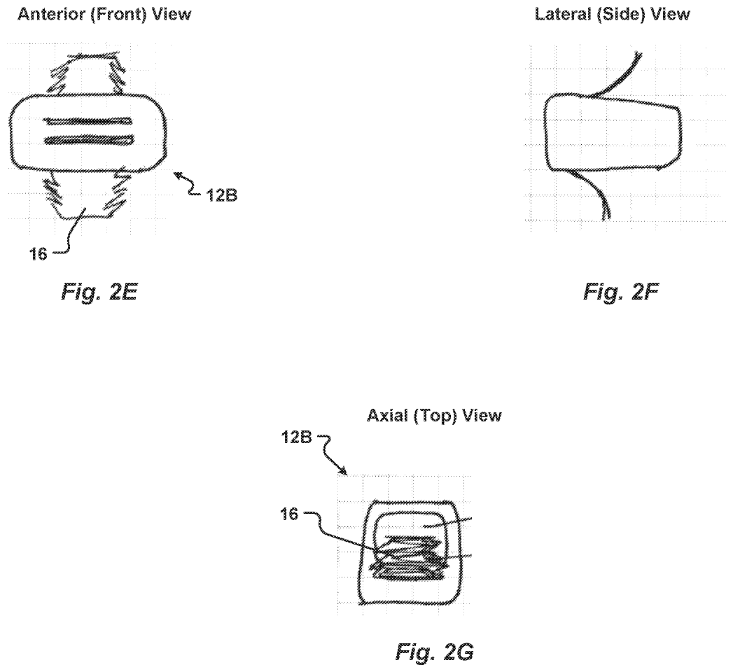

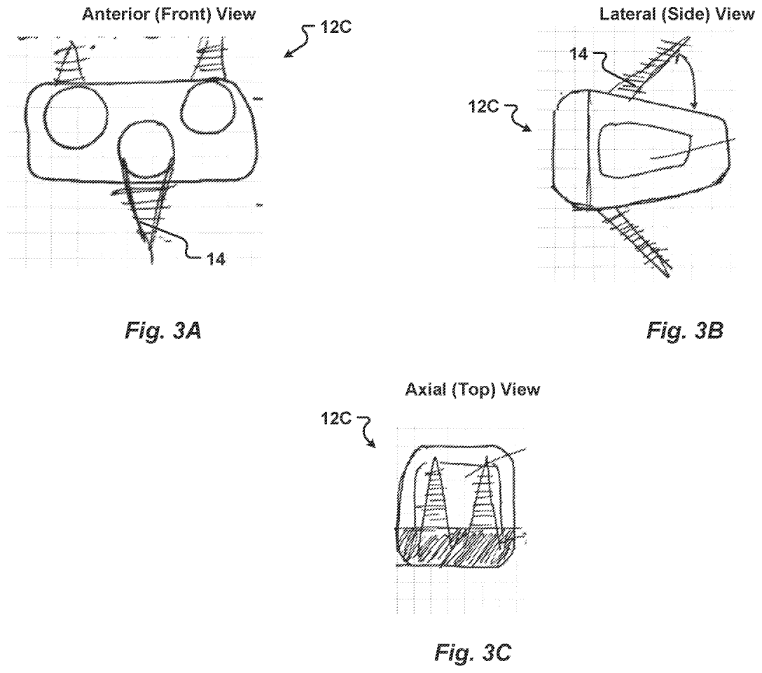

The drawings are not necessarily (but may be) to scale. In certain instances, details that are not necessary for an understanding of the disclosure or that render other details difficult to perceive may have been omitted. It should be understood, of course, that the disclosure is not necessarily limited to the embodiments illustrated herein. As will be appreciated, other embodiments are possible using, alone or in combination, one or more of the features set forth above or described below. For example, it is contemplated that various aspects, features and devices shown and/or described with respect to one embodiment may be combined with or substituted for aspects, features or devices of other embodiments regardless of whether or not such a combination or substitution is specifically shown or described herein.

The following is a listing of components according to various embodiments of the present disclosure, and as shown in the drawings:

| Number | Component |
|---|---|
| 2 | Vertebrae |
| 4 | Groove or slot |
| 6 | Disc space |
| 8 | Plate |
| 10 | Plate screw |
| 12 | Cage |
| 14 | Cage screw |
| 16 | Blade or shim |
| 18 | Disc replacement device |
| 20A | Cranial (upper) endplate |
| 20B | Caudal (lower) endplate |
| 22 | Medial element |
| 24 | Keel |
| 26 | Teeth |
| 30 | Interbody device |
| 32 | Median plane |
| 34 | Endplate |
| 34A | Superior endplate |
| 34B | Inferior endplate |
| 36 | Orifice in endplate |
| 38 | Strut |
| 40 | Dome |
| 42 | Anterior surface |
| 44 | Engagement feature |
| 46 | Support |
| 48 | Posterior surface |
| 50 | Lateral surface |
| 52 | Apertures in lateral surface |
| 54 | Friction element |
| 56 | Keel |
| 58 | Projection |
| 60 | Teeth |
| 62 | Interior cavity |
| 130 | Interbody device |
| 131 | Coronal (or vertical) plane |
| 132 | Median plane |
| 133 | Transverse (or horizontal) plane |
| 134 | Endplate |
| 134A | Superior endplate |
| 134B | Inferior endplate |
| 136 | Orifice in endplate |
| 142 | Anterior wall |
| 143 | Height of anterior wall |
| 144 | Engagement feature |
| 148 | Posterior wall |
| 149 | Height of posterior wall |
| 150 | Lateral plate |
| 152 | Apertures in lateral plate |
| 154 | Friction element |
| 156 | Anterior end of friction element |
| 158 | Posterior end of friction element |
| 160 | Teeth |
| 162 | Interior cavity |
| 164 | Corner |
| 166 | Angle of friction element |
| 168 | Angle of lateral plate |
| 170 | Aperture of engagement feature |
| 172 | Thread of engagement feature |
| 174 | Maximum width |
| 176 | Minimum width |
| 178 | Depth of interbody device |
| 180 | Length of a friction element |
| 182 | First side of tooth |
| 184 | Long side of tooth |
| 186 | Fixed end of tooth |
| 188 | Free end of tooth |
| 190 | Height of tooth |
| 192A | First plane |
| 192B | Second plane |
| 194 | Angle of endplate |
| 196 | Maximum height of interbody device |
| 198 | Maximum distance between teeth |

### DETAILED DESCRIPTION

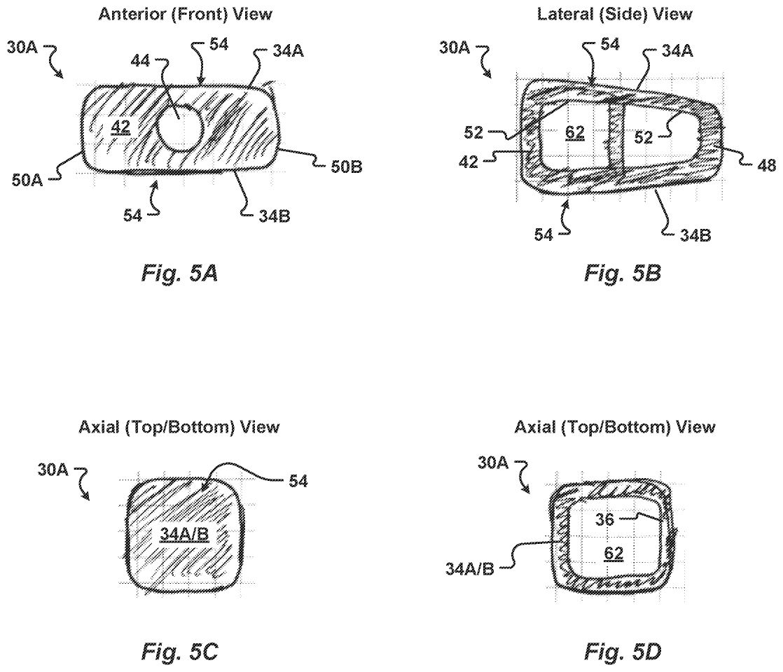

Referring now to FIGS. 5A-5D, an embodiment of an interbody device 30A of the present disclosure is generally illustrated. The interbody device 30 includes superior and inferior endplates 34A, 34B, an anterior surface 42, a posterior surface 48, and lateral surfaces 50. Friction elements 54 are formed on the endplates 34. The friction elements 54 are configured to engage a superior vertebral body above the interbody device and an inferior vertebral body below the interbody device. In some embodiments, the friction elements comprise one or more of a bump, a ridge, a post, a point, a hook, a tooth, or other projections that extend from the endplates to frictionally engage a vertebral body. In this manner, the friction elements 54 prevent inadvertent or unintended movement of the interbody device 30 relative to the vertebral bodies.

The friction elements provide a pull out strength that is at least equal to the screws 14 or shims 16 used with cages 12A, 12B. One of skill in the art will appreciate that the interbody device 30 of the present disclosure can be placed in a disc space quicker and with less risk than cages that require screws for retention because no screws are required to anchor the interbody device into the vertebral bodies. Moreover, the friction provided by the friction elements 54 eliminates the need to install a plate into the superior and inferior vertebrae as required with cages. By eliminating the plate, the interbody device 30 of the present disclosure further reduces the risk to the patient and the possibility of damage to the vertebrae. For example, screws can damage the vertebrae and may weaken the vertebrae. Further, screws can become loose or move relative to the vertebrae. Eliminating the plate also reduces the time required to plan and perform the surgery which is beneficial to both the patient, the surgeon, and the medical facility, and reduces the material costs associated with the plate and screws used in the surgery. Additionally, by facilitating faster surgeries, the interbody devices of the present disclosure frees operating rooms for other procedures.

Optionally, one or more surface (such as an endplate 34, the anterior surface 42, the posterior surface 48, and a lateral surface 50) of the interbody device 30 are treated to facilitate bone in-growth. In this manner, the interbody device 30 of the present disclosure can optionally be used without inserting bone graft material into the disc space. This is beneficial because bone graft material is expensive and some commercially supplied bone graft materials can lead to complications.

In some embodiments, a surface of the interbody device 30 has a micro-porous structure for bone ingrowth or adherence. In another embodiment, one or more of the surfaces comprises a mesh. A surface of the interbody device 30 may include a plurality of apertures such that it is between approximately 10% open and approximately 90% open. In embodiments, the superior endplate 34A is between approximately 10% open and approximately 90% open. Additionally, or alternatively, the inferior endplate 34B is between approximately 10% open and approximately 90% open.

In embodiments, the lateral surfaces 50A, 50B are approximately parallel. However, other configurations are contemplated in which the lateral surfaces are not parallel. The lateral surfaces 50 can include an aperture 52 to access an interior cavity 62 within the interbody device. Accordingly, the interior cavity 62 can optionally be filled with bone graft material to facilitate fusion of the adjacent vertebrae. However, the interbody device 30 can optionally be positioned in the disc space without inserting either bone graft material or a bone graft substitute within the disc space.

In some embodiments, the anterior surface 42 has a height of between approximately 5 mm and approximately 12 mm, or approximately 8 mm. In other embodiments, the anterior surface 42 has a width of between approximately 10 mm and approximately 22 mm, or approximately 16 mm.

The superior endplate 34A is oriented in a diverging relationship to the inferior endplate 34B in embodiments of the present disclosure. More specifically, in embodiments, the endplates 34 are not parallel and are oriented at a predetermined angle. Optionally, one or more of the endplates 34 is generally planar.

Referring now to FIG. 5C, one or more of the superior endplate 34A and the inferior endplate 34B extend continuously between the lateral surfaces. Additionally, or alternatively, one or more of the endplates 34 can include an orifice 36 such as generally illustrated in FIG. 5D.

Optionally, the anterior surface 42 includes an engagement feature 44. The engagement feature 44 is adapted to interface with a surgical tool, such as an inserter, used to position the interbody device 30 within the disc space. In some embodiments, the engagement feature 44 comprises an aperture or bore in the anterior surface. The engagement feature 44 may be generally circular. Threads may optionally be formed in the engagement feature 44 to releasably connect the interbody device to the surgical tool. Other configurations and locations for the engagement feature are contemplated. More specifically, the interbody device 30 of the present disclosure can include an engagement feature 44 configured to connect to any prior art surgical tool or insertion device for use in a fusion procedure.

The interbody devices 30 of all embodiments of the present disclosure can be made of any suitable material. In embodiments, the interbody device 30 optionally comprises a plastic. The plastic may be a thermoplastic polymer. Optionally, the plastic is a polyether ether ketone (PEEK). Additionally, or alternatively, in other embodiments, the interbody device optionally comprises a metal. In some embodiments, the metal is titanium or a titanium alloy.

In embodiments, the interbody device 30 is produced as a casting. Optionally, the interbody device 30 is forged. Additionally, or alternatively, the interbody device 30 may be produced or finished by an additive manufacturing process. In some embodiments, the interbody device is produced by a 3-D printing process. In embodiment, the interbody device is treated to produce micro- and nanoscale surface roughness. For example, the interbody device 30 may be formed by casting and/or forging and the micro- or nanoscale roughness is created with a 3-D additive process. In any embodiment, the treatment of the interbody device optionally includes acid etching to promote the micro- and nanoscale surface roughness. Additionally, or alternatively, the interbody device is heat treated to promote the micro- and nanoscale surface roughness.

Notably, unlike the related disc replacement devices 18, the interbody device is rigid. In embodiments, the interbody device is of a one-piece construction. For example, in some embodiments the interbody device has no moving parts or pieces.

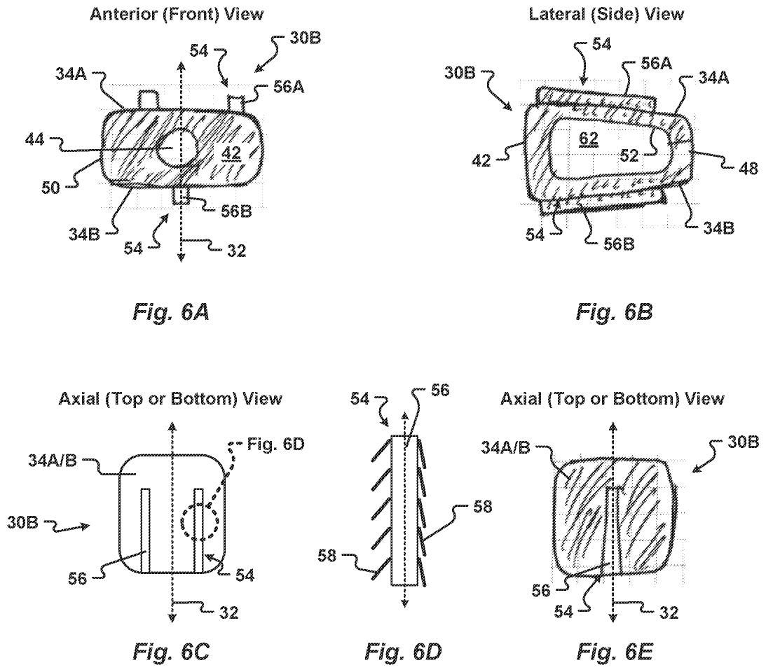

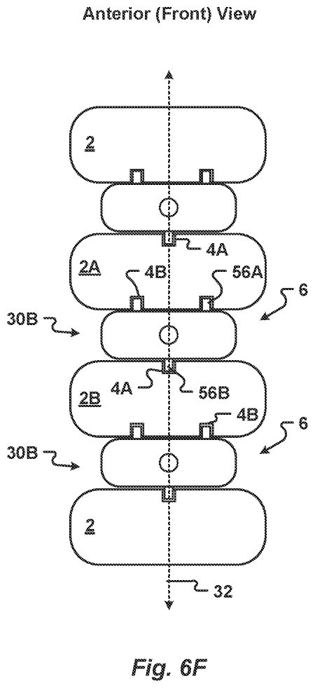

Referring now to FIGS. 6A-6E, another embodiment of an interbody device 30B of the present disclosure is generally illustrated. The interbody device 30B can include any or all of the features of the interbody device 30A. Moreover, the interbody device 30B may be formed of the same or similar materials as the interbody device 30A.

Notably, the friction element 54 of the interbody device includes keels 56 that extend from the endplates. Optionally, the keels 56 are generally linear. The keels 56 extend generally parallel to a median plane 32 that bisects the interbody device. As generally shown in FIGS. 6C, 6E, the keels extend along a predetermined length of the endplates. In some embodiments, a keel 56 extends between about 50% and about 100% of the length of the endplate.

In embodiments, a keel of the present disclosure has a height of less than about 2 mm. For example, the height of the keel can be about 1.5 mm, or less.

Optionally, the keel 56 includes a micro-porous surface to encourage bone in-growth.

Referring now to FIG. 6D, the keels 56 optionally include projections to engage a vertebral body. The projections 58 can extend from the lateral surfaces and/or the axial surface of the keel.

In some embodiments, the projections are flexible. For example, the projections can be configured to bent or pivot into a first position during placement of the interbody device in the disc space. In the first position, the projections provide minimal resistance to movement in a first direction relative to the vertebral body, such as when the interbody device is pushed in a posterior direction into the disc space. Continuing this example, the projections can move to a second position to increase resistance to movement in a second direction, such as to resist movement in an axial direction out of the disc space. In embodiments, the projections are integrally formed with the keel and the interbody device 30B.

In embodiments, a projection has a first end fixed to a lateral surface of a keel as generally illustrated in FIG. 6D. A second end of the projection extends away from the keel. One embodiment of the first position of the projections is generally illustrated on the right side of the keel in FIG. 6D. In the first position, the second end of the projection is positioned proximate to the lateral surface of the keel. The second position of the projections is shown according to another embodiment on the left side of FIG. 6D. In some embodiments, in the second position the second end of the projection is positioned distal to the lateral surface of the keel.

The endplates 34A, 34B can include any number of keels 56. In some embodiments, the superior endplate 34A includes 1 or 2 keels 56A. The inferior endplate 34B can also include 1 or 2 keels 56B. However, in other embodiments, the inferior endplate 34B has a different number of keels than the superior endplate. For example, in some embodiments, the superior endplate has two keels and the inferior endplate has one keel. In other embodiments, the superior endplate has one keel and the inferior endplate has two keels.

Other configurations of the keels are contemplated.

Referring now to FIG. 6F, in use, a groove or slot 4 can optionally be formed in the superior and inferior vertebrae 2A, 2B to receive the keels 56 of the interbody device 30B. The groove 4 can be formed in any suitable manner known to those of skill in the art. In some embodiments, the keel 56 of the interbody device 30B is adapted to cut or form a slot 4 in the vertebral body during insertion into the disc space.

Notably, in embodiments, the keels 56A of the superior endplate 34A are offset relative to the medial plane 32 from the keels 56B of the inferior endplate 34B. More specifically, in embodiments, the keels of the endplates 34 are not coplanar. This is beneficial when the interbody device 30 is positioned at multiple adjacent levels of a patient's spine as generally illustrated in FIG. 6F. Because the superior keels 56A are offset from the inferior keels 56B, if grooves are formed in a vertebrae between two interbody devices, the superior groove 4A in the vertebrae will also be offset from the inferior grooves 4B as shown in vertebrae 2A, 2B. Offsetting the superior and inferior grooves 4A, 4B in the vertebrae reduces the risk of damaging the vertebrae.

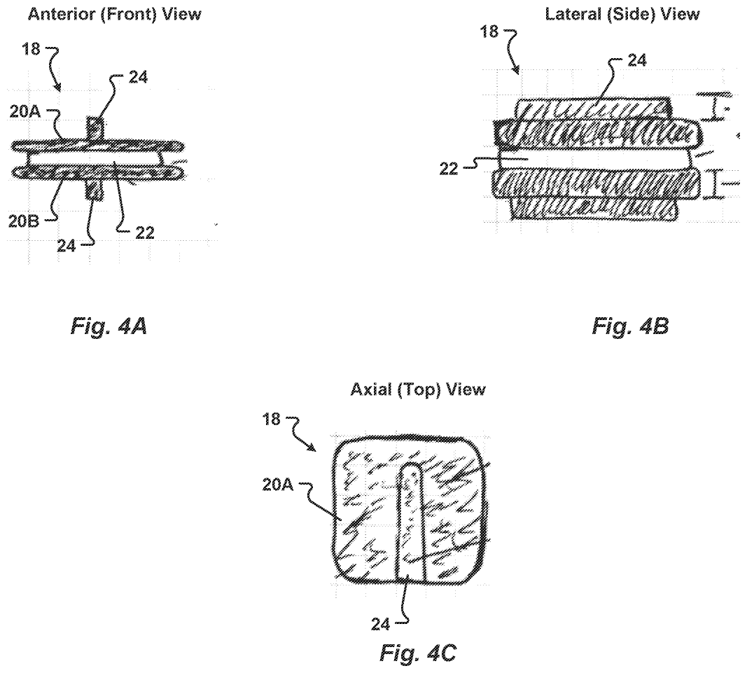

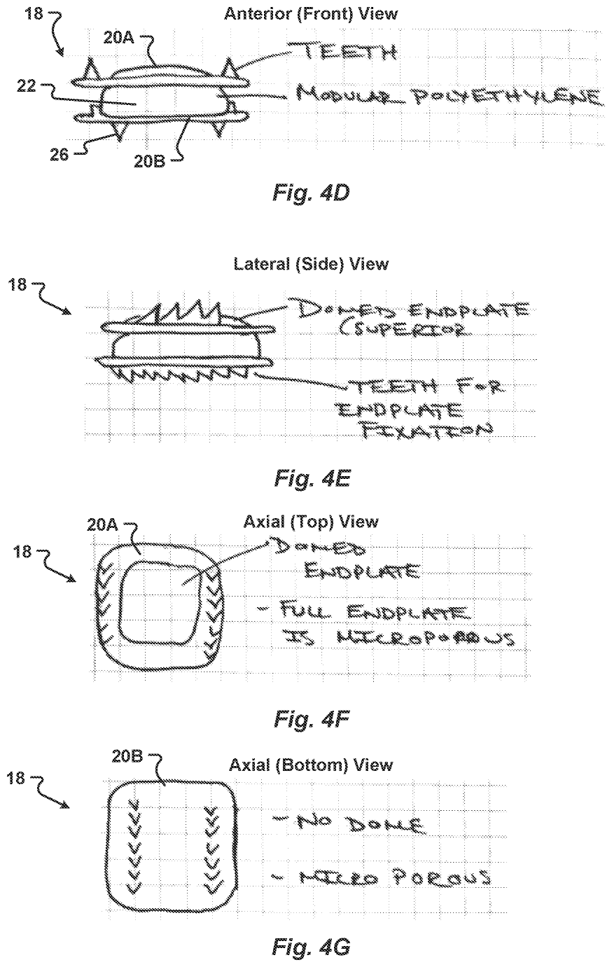

In contrast, the keels 24 of the disc replacement device 18 described in conjunction with FIG. 4 are generally coplanar. Accordingly, grooves formed in vertebrae to receive the disc replacement device will also be coplanar. When a groove is formed in a superior surface of a vertebrae that is aligned (or coplanar) with a groove formed in an inferior surface of the vertebrae, the aligned grooves combine to reduce the thickness of the vertebrae in a plane. This can weaken the superior and inferior vertebrae when disc replacement devices are positioned at several levels. For example, the vertebrae may crack or fracture between two coplanar grooves.

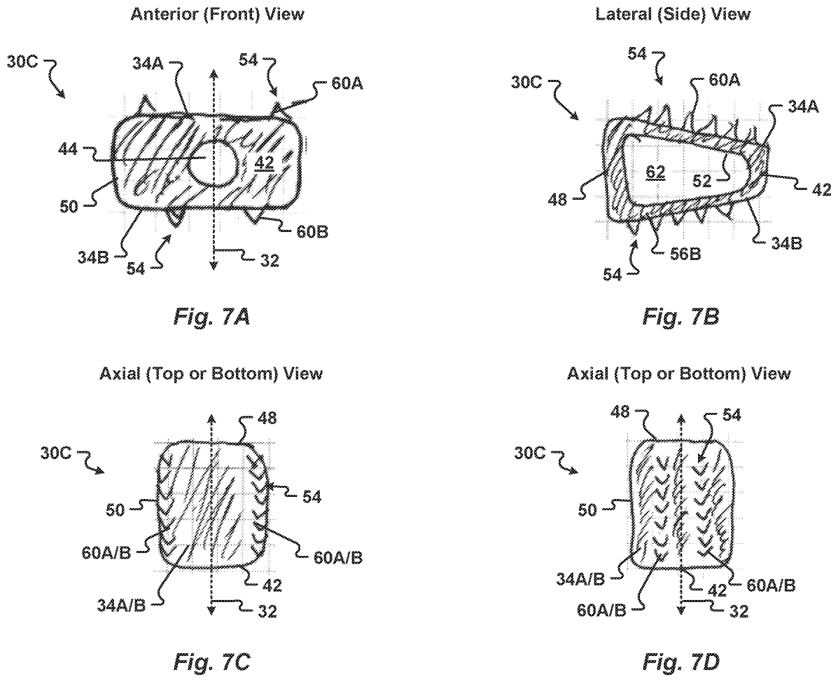

Referring now to FIG. 7, still another embodiment of an interbody device 30C of the present disclosure is generally illustrated. The interbody device 30C includes many of the same, or similar, features as the interbody devices 30A, 30B.

The interbody device 30C has a friction element 54 which comprises teeth 60. The teeth 60 are similar to the keels 56 and can have a similar arrangement and position on the endplates 34 of the interbody device 30C.

In any of the embodiments, the teeth 60 are optionally formed in a row that extends approximately parallel to a median plane 32 that bisects the interbody device. The rows extend a predetermined length of the endplates. In some embodiments, a row of teeth 60 extends at least 50% of the length of the endplate. Optionally, the row of teeth can extend from proximate to the anterior surface 42 to proximate to the posterior surface 48.

The teeth 60 generally include a free end that projects away from an associated endplate. In embodiments, the teeth have a fixed end engaged with an endplate that is wider than the free end. Optionally, the free end is pointed.

In embodiments, the free end of one or more of the teeth is oriented toward the anterior surface 42 of the interbody device. Additionally, or alternatively, one or more of the teeth have a free end that is oriented toward the posterior surface 48 of the interbody device.

Optionally, the teeth in a row of teeth have free ends that are spaced two or more distances from median plane. More specifically, a first tooth in a row can have a free end that is spaced a first distance laterally from the median plane. A second tooth in the row may have a free end that is spaced a second distance laterally from the median plane. In this manner, the free ends of the teeth are not coplanar to increase pull out resistance of the interbody device. Accordingly, in some embodiments, a first tooth in a row of teeth 60 has a free end oriented toward a first lateral surface 50 of the interbody device. A second tooth in the row of teeth 60 has a free end oriented toward a second lateral surface of the interbody device.

In some embodiments, the teeth are rigid. Alternatively, the teeth are flexible. The teeth can be integrally formed with the interbody device 30C.

Optionally, the teeth are configured to permit movement in a first direction (such as in the posterior direction when the interbody device is being positioned in the disc space) and resist movement in a second direction (for example, in the anterior direction). In embodiments, the teeth can bend or flex into a retracted positioned during movement in the first direction. Additionally, or alternatively, the teeth can bend or flex into an extended position during movement of the interbody device in the second direction.

The endplates 34A, 34B can have any number of rows of teeth 60. In some embodiments, the superior endplate 34A includes 1 or 2 rows of teeth 60A. The inferior endplate 34B can also include 1 or 2 rows of teeth 60B. However, in embodiments, the inferior endplate 34B has a different number of rows of teeth than the superior endplate. For example, in some embodiments, the superior endplate has two rows of teeth and the inferior endplate has one row of teeth. In other embodiments, the superior endplate has one row of teeth and the inferior endplate has two rows of teeth. Optionally, the superior endplate and the inferior endplate both have two rows of teeth.

In one configuration, the superior endplate 34A has two rows of teeth 60A that are positioned proximate to the lateral surfaces 50 of the interbody device. Alternatively, the two rows of teeth of the superior endplate are spaced from the lateral surfaces of the interbody device.

Optionally, the inferior endplate 34B has two rows of teeth 60B that are positioned proximate to the lateral surfaces 50 of the interbody device. Alternatively, the two rows of teeth of the inferior endplate are spaced from the lateral surfaces of the interbody device.

Similar to the keels of the interbody device 30B, the teeth 60A of the superior endplate 34A are optionally offset medially from the teeth 60B of the inferior endplate as generally illustrated in FIG. 7A. As described above in conjunction with FIGS. 6A-6F, this is beneficial because if grooves 4A, 4B are formed in superior and inferior surfaces of a vertebral body to receive rows of teeth of two interbody devices, the superior grooves 4A will be offset from the inferior grooves 4B.

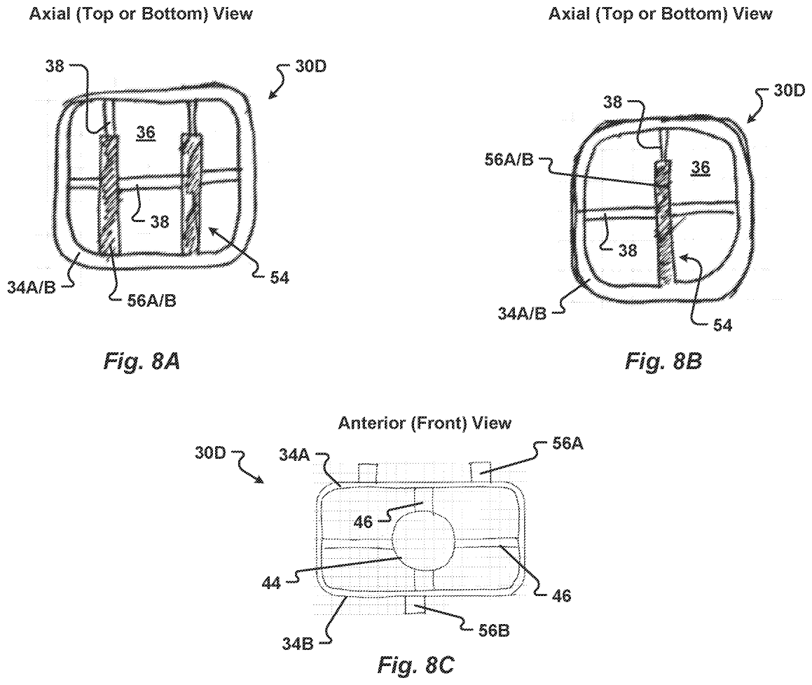

Referring now to FIG. 8, another embodiment of an interbody device 30D of the present disclosure is illustrated. The interbody device 30D is similar to interbody devices 30A-30C. Notably, one or more of the superior endplate 34 and the inferior endplate 34 is substantially open.

The interbody device 30D includes one or more keels 56. The keels are supported by a strut 38.

Additionally, or alternatively, the interbody device 30D can also include an anterior surface 42 and/or a posterior surface (not illustrated) that is substantially open. In some embodiments, the engagement feature 44 is fixed to a support 46.

The open surfaces of the endplates and the anterior/posterior surfaces facilitate the optional packing an interior cavity 62 of the interbody device with bone graft material.

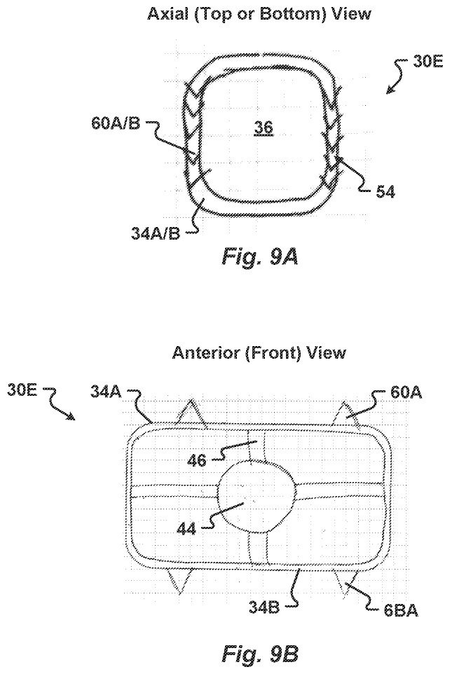

Yet another embodiment of an interbody device 30E of the present disclosure is shown in FIG. 9. The interbody device 30E is similar to the interbody device 30D with one or more endplate 34A, 34B that is substantially open. However, the interbody device 30E includes teeth 60 similar to the interbody device 30C.

Additionally, or alternatively, the interbody device 30E can also include an anterior surface 42 and/or a posterior surface (not illustrated) that is substantially open. In some embodiments, the engagement feature 44 is fixed to a support 46.

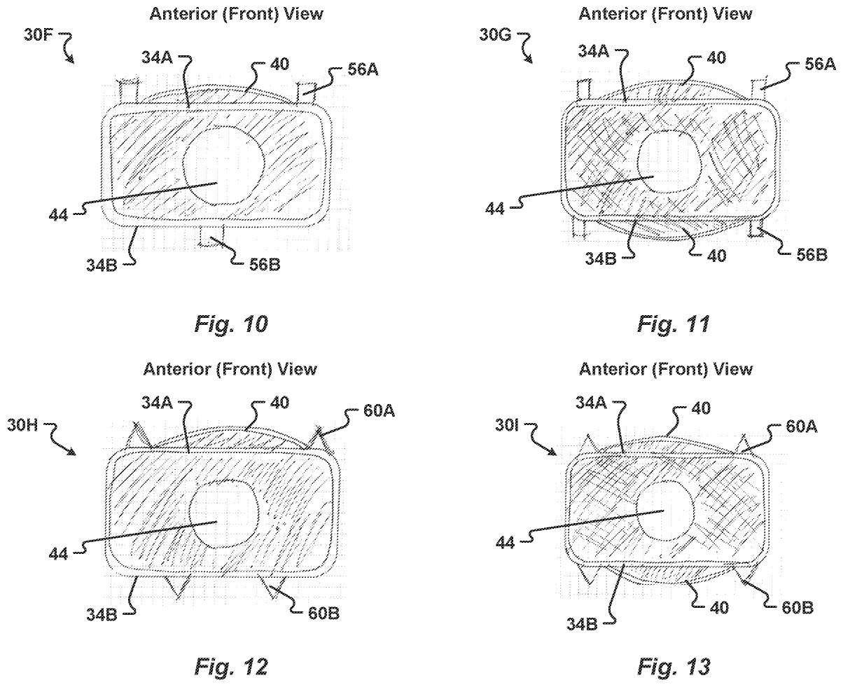

Referring now to FIGS. 10-13, embodiments of interbody devices 30 with one or more domes 40 are generally illustrated. More specifically, embodiments of the interbody devices of the present disclosure can have a dome that extends from at least one of the superior endplate 34A and the inferior endplate 34B. In one configuration, the interbody devices 30 have one dome. Optionally, the interbody devices 30 have two domes 40.

The domes 40 are configured to fit into a concave surface of a superior or inferior vertebral body facing a disc space. More specifically, the domes 40 are anatomically shaped to engage the concave surfaces of vertebral bodies. The dome 40 increases the surface area of the interbody device engaged with the vertebral body. In this manner, the dome 40 increases the pull-out resistance of the interbody device.

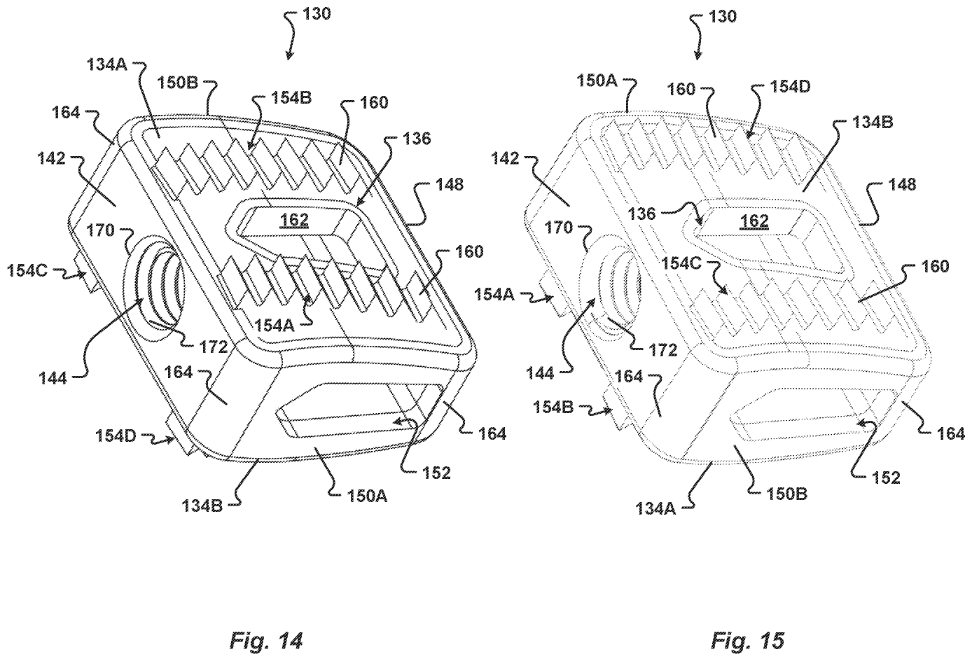

Referring now to FIGS. 14-23, anther embodiment of an interbody device 130 of the present disclosure is generally illustrated. The interbody device 130 is similar to other embodiments of interbody devices 30 described herein and includes many of the same or similar features.

The interbody device 130 is adapted for use in ACDF and ALIF procedures. It includes features to frictionally engage an upper vertebral body and a lower vertebral body. Moreover, the interbody device 130 is configured to provide sufficient friction (or pull-out resistance) once positioned in a disc space such that deployable fixation devices (e.g., screws, pins, rods, anchors, plates and the like) are not required for use with the interbody device. In some embodiments, the interbody device 130 uses static friction elements 154 to engage the adjacent vertebral bodies. Optionally, for any of the embodiments, the interbody device 130 is wholly static, meaning it does not include moving parts, deployable anchors (such as screws, pins, and the like), or interaction with screws or other fixation devices to reduce (or prevent) unintended or inadvertent movement.

The interbody device generally includes a superior endplate 134A opposite to an inferior endplate 134B, an anterior wall 142 opposite to a posterior wall 148, lateral plates 150 extending between the endplates 134A, 134B, an engagement feature 144, and friction elements 154. In some embodiments, corners 164 between the lateral plates 150 and the anterior and posterior walls 142, 148 are optionally rounded with a predetermined radius of curvature. Although described herein as a superior endplate 134A and an inferior endplate 134B, in use the interbody device 130 can be positioned within a disc space with either endplate 134A, 134B oriented cranially.

An orifice 136 may be formed through one or more of the superior and inferior endplates 134A, 134B. Additionally, or alternatively, an aperture 152 may also be formed through one or more of the lateral plates 150. The orifice 136 and/or the aperture 152 may extend to an interior cavity 162 formed within the interbody device 130. In this manner, the interior cavity 162 can optionally be filled with bone graft material during a surgical procedure to facilitate fusion of the adjacent vertebrae. Further, after the interbody device 130 is positioned in a patient's disc space, the bone graft material can flow out of the interior cavity 162 outwardly through one or more of the orifice 136 and the aperture 152 and into the disc space. However, the interbody device 130 can optionally be positioned in a disc space without inserting either bone graft material or a bone graft substitute within the disc space or the interior cavity 162.

The orifice 136 and the aperture 152 may have any size or shape. Optionally, for any of the embodiments, one or more of the orifice 136 and the aperture 152 have a shape that is generally pentagonal. The orifice and the aperture may have sizes selected to meet minimum requirements for access to the interior cavity 162. More specifically, the orifice 136 has a size selected to maximize a surface area of the endplate 134. As will be understood by one of skill in the art, the endplates 134 will frictionally engage superior or inferior surfaces of adjacent vertebrae and increase the pull-out strength of the interbody device. Accordingly, the orifice 136 is adapted to provide access to the interior cavity 162 without unnecessarily decreasing the surface area of the endplates 134 below a predetermined surface area.

Optionally, for any of the embodiments, one or more of the endplates 134A, 134B are adapted to facilitate osseointegration between the interbody device 130 and adjacent vertebrae. For example, one or both of the endplates 134 can include micro porous areas to allow for bone in-grow. One or more surfaces of the interbody device 130 may include a plurality of apertures such that the surface is between approximately 10% open and approximately 90% open. In embodiments, one or more of the superior endplate 134A and the inferior endplate 134B is between approximately 10% open and approximately 90% open.

Any suitable method of enabling osseointegration between the vertebrae and the interbody device 130 known to those of skill in the art may be used. In some embodiments, at least a portion of each endplate 134 is porous to facilitate bone growth and integration with boney endplate of the adjacent vertebrae. The anterior wall 142, posterior wall 148, and lateral plates 150 may also be adapted for osseointegration.

A friction element 154 extends from one or more of the endplates 134. The friction element is configured to increase the pull-out resistance of the interbody device and to prevent or reduce unintended or inadvertent movement of the interbody device. The endplates 134A, 134B can have any number of friction elements 154. In some embodiments, the interbody device has from two to six friction elements 154. Optionally, for any of the embodiments, the friction elements 154 are static and do not move relative to the interbody device 130.

In some embodiments, the superior endplate 134A includes one or two friction elements 154. The inferior endplate 134B can also include one or two friction elements 154. However, in some embodiments, the inferior endplate 134B has a different number of friction elements 154 than the superior endplate. For example, in embodiments, the superior endplate has two friction elements 154 and the inferior endplate has one friction element 154. In other embodiments, the superior endplate has one friction element 154 and the inferior endplate has two friction elements. Optionally, the superior endplate and the inferior endplate both have two friction elements 154. In embodiments, the superior endplate has from two to six friction elements 154. Additionally, or alternatively, the inferior endplate optionally includes from two to six friction elements 154.

In some embodiments, the interbody device 130 has four friction elements 154A, 154B, 154C and 154D. Optionally, two friction elements 154A, 154B extend from the superior endplate 134A and two friction elements 154C, 154D extend from the inferior endplate 134B.

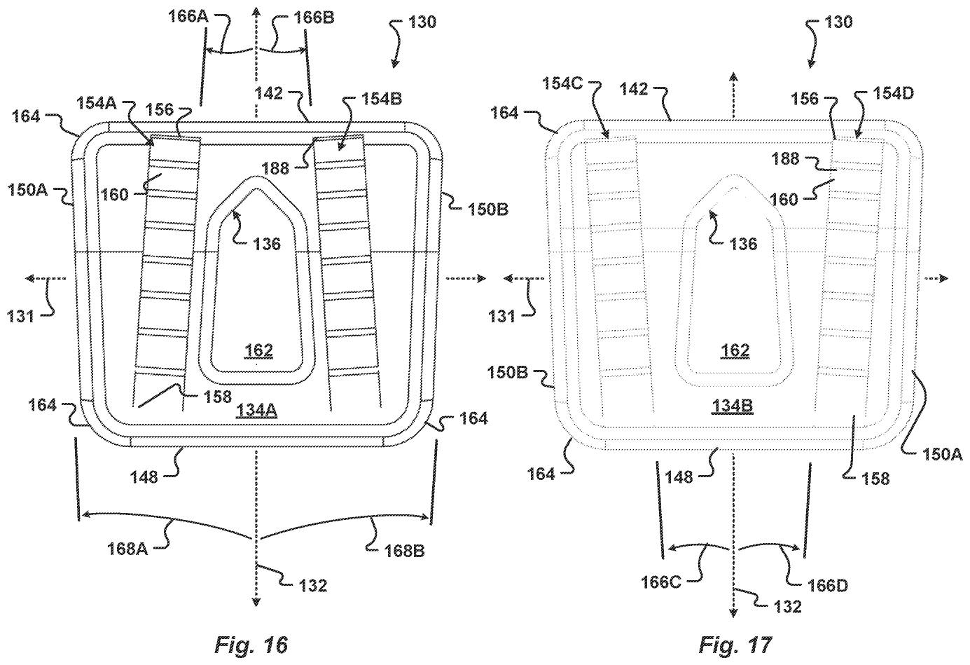

Referring now to FIGS. 16 and 17, each friction element is oriented at a predetermined angle 166 relative to a median plane 132 that bisects the interbody device 130 and that extends through the endplates 134A, 134B. The median plane 132 is substantially perpendicular to the plane of FIG. 16. Optionally, for any of the embodiments, one or more of the friction elements 154 is oriented at an oblique angle relative to the median plane 132. In this manner, the friction elements 154 increase the force necessary to pull (or move) the interbody device in the anterior direction (or in a direction parallel to the median plane 132) when the interbody device is positioned in a disc space of a patient.

In some embodiments, the first and second friction elements 154A, 154B are optionally oriented at an oblique angle to each other. Additionally, or alternatively, the third friction element 154C may optionally be oriented at an oblique angle to the fourth friction element 154D.

The friction elements 154A, 154B of the superior endplate 134A may be oriented such that anterior ends 156 of each friction element 154A, 154B are a first distance from the median plane 132. In some embodiments, the posterior ends 158 are a second distance from the median plane that is greater than the first distance.

In contrast, the friction elements 154C, 154D of the inferior endplate 134B can be oriented such that anterior ends 156 of each friction element 154C, 154D are a third distance from the median plane 132. The posterior ends 158 of friction elements 154C, 154D are a fourth distance from the median plane. Optionally, in some embodiments, the third distance is greater than the fourth distance.

In some embodiments, the first friction element 154A is approximately parallel to the third friction element 154C. Additionally, or alternatively, the second friction element 154B may be approximately parallel to the fourth friction element 154D.

Referring now to FIG. 16, in embodiments, the first friction element 154A is oriented at a first angle 166A and the second friction element 154B is oriented at a second angle 166B with respect to the median plane 132. In some embodiments the first and second angles are of approximately the same magnitude, but the first angle 166A is a negative angle and the second angle 166B is a positive angle in the perspective of FIG. 16.

The first angle 166A of the first friction element is optionally between about −3.0° and about −5.0°, or about −3.75° relative to the median plane 132. In some embodiments, the second angle 166B of the second friction element is between about 3.0° and about 5.0°, or about 3.75° relative to the median plane 132.

Similarly, as generally shown in FIG. 17, the third friction element 154C may be oriented at a third angle 166C and the fourth friction element 154D can be oriented at a fourth angle 166D relative to the median plane 132. In some embodiments the third and fourth angles are of approximately the same magnitude, but the third angle 166C is a positive angle and the fourth angle 166D is a negative angle in the perspective of FIG. 17.

Optionally, the third angle 166C of the third friction element is oriented at between about 3.0° and about 5.0°, or about 3.75° relative to the median plane 132. In some embodiments, the fourth angle 166D of the fourth friction element 154D is between about −3.0° and about −5.0°, or about −3.75° relative to the median plane 132.

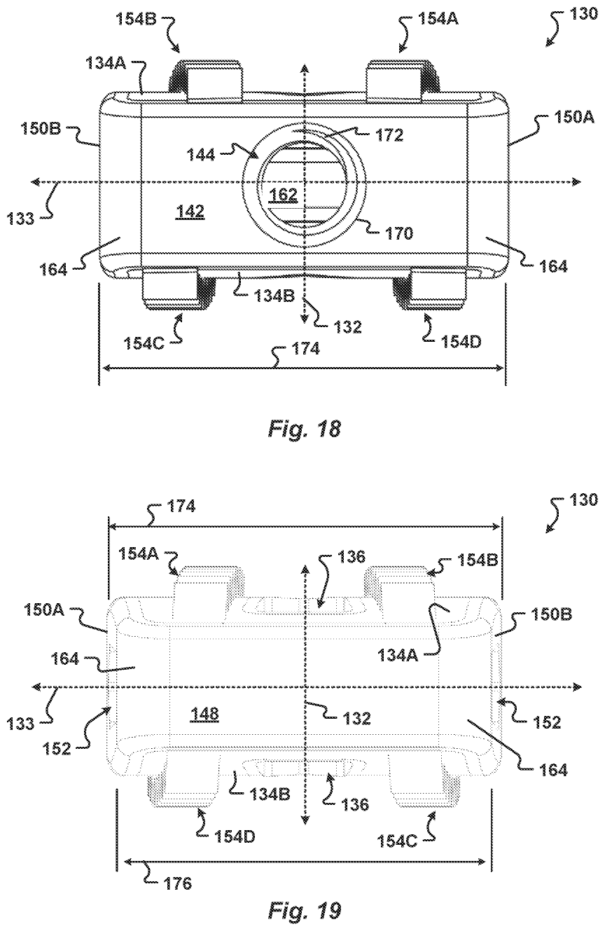

These orientations of the friction elements 154 beneficially offset the friction elements 154A, 154B of the superior endplate in the vertical dimension from the friction elements 154C, 154D of the inferior endplate. For example, as generally illustrated in FIGS. 18, 19, the friction elements 154A, 154D are not coplanar. Similarly, friction elements 154B, 154C are not coplanar. This is beneficial because when a first interbody device 130 is positioned cranially relative to a superior surface of a vertebral body and a second interbody device 130 is positioned caudally relative to an inferior surface of the vertebral body, the friction elements will contact the vertebral body along four different planes to distribute forces applied to the vertebral body.

As generally illustrated in FIGS. 16, 17, the lateral plates 150A, 150B of the interbody device are optionally oriented at oblique angles 168 relative to the median plane 132. Accordingly, the anterior wall 142 has a first width that is greater than a second width of the posterior wall 148. In this manner, the interbody device 130 is configured with a geometry that approximates the shape of a disc to be replaced. In some embodiments, the first lateral plate 150A is oriented at an angle 168A of between about 1.20° and about 3.20°, or about 2.20° relative to the median plane 132. Similarly, the second lateral plate 150B may be oriented at an angle 168B of between about −1.20° and about −3.20°, or about −2.20° relative to the median plane 132.

Referring now to FIGS. 18 and 19, the anterior 142 and posterior 148 walls are generally illustrated. Optionally, for any of the embodiments, the anterior wall 142 is approximately parallel to the posterior wall 148.

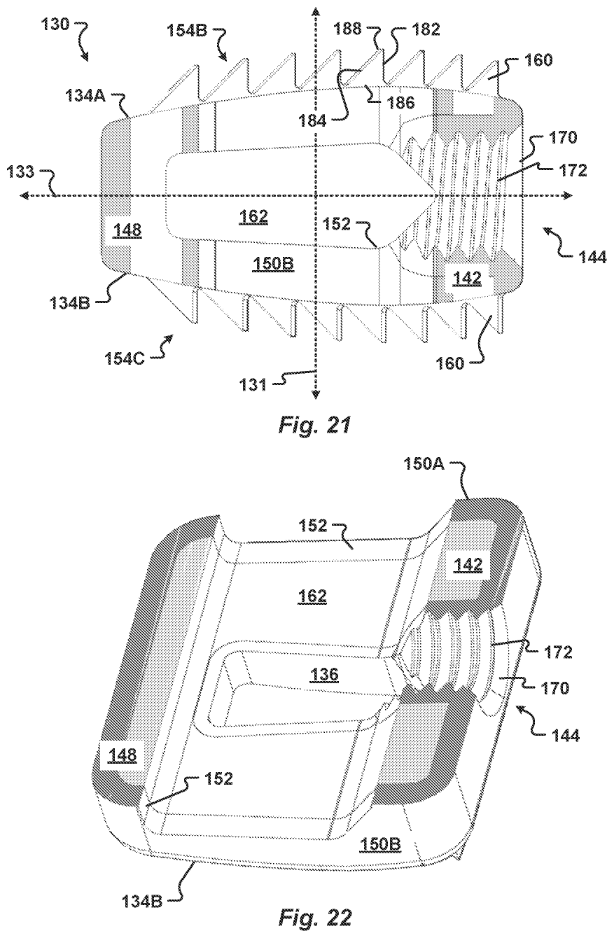

Notably, the anterior wall 142 has an engagement feature 144. The engagement feature is adapted to interface with a surgical tool, such as an inserter, used to position the interbody device 130 within the disc space during a surgical procedure. Optionally, for any of the embodiments, the engagement feature 144 comprises an aperture or bore 170 extending at least partially into the anterior wall 142. The aperture 170 may optionally extend through the anterior wall 141 and intersect an interior cavity 162 of the interbody device (as generally illustrated in FIGS. 21 and 22). Accordingly, in some embodiments, bone graft material may be introduced into the interior cavity 162 through the aperture 170 of the engagement feature 144. The aperture is optionally generally circular.

Optionally, for any of the embodiments, a thread 172 is formed in the engagement feature aperture 170 to releasably connect the interbody device 130 to the surgical tool. The aperture 170 and thread 172 may have any size and configuration. In embodiments, the aperture 170 has a diameter of between about 3 mm and about 6 mm, or about 4 mm. Additionally, or alternatively, the thread 172 optionally has a pitch of one thread per 0.7 mm. Accordingly, in some embodiments, the engagement feature 144 is configured to engage a tool with an M4×0.7 thread such as known to those of skill in the art.

Other configurations and locations for the engagement feature 144 are contemplated. More specifically, the interbody device 130 of the present disclosure can include an engagement feature 144 configured to connect to any prior art surgical tool or insertion device for use in a fusion procedure.

FIG. 18 also illustrates a maximum width 174 of the lateral plates 150 of the interbody device. The maximum width 174 is proximate to the anterior wall 142. Optionally, for any of the embodiments, the maximum width 174 is between about 12.0 mm and about 17.0 mm, or about 14.9 mm.

The minimum width 176 of the lateral plates 150 is generally illustrated in FIG. 19. In some embodiments, the minimum width 176 is between about 11.0 mm and about 16.0 mm, or about 14.2 mm.

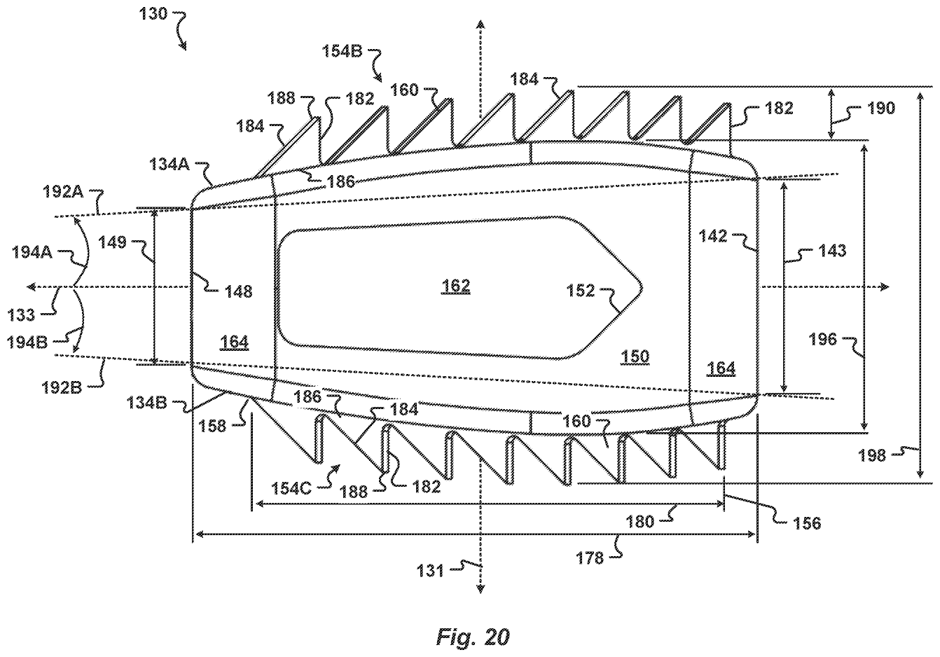

Referring now to FIG. 20, each friction element 154 extends a predetermined length 180. In embodiments, the length 180 of a friction element extends at least 50% of a depth 178 of the interbody device 130. In other embodiments, a friction element extends between about 60% and about 98% of the device depth 178. Optionally, the length 180 of the friction element 154 is between about 9.0 mm and about 13.0 mm, or about 10.9 mm. Optionally, for any of the embodiments, the depth 178 of the interbody device is between about 10.0 mm and about 16.0 mm, or about 13.0 mm.

In some embodiments, the friction elements 154 comprises a keel 56 as described herein. Optionally, the friction element 154 includes a projection 58 as described herein. The projection may be flexible or otherwise moveable.

In embodiments, one or more of the friction elements 154 extends along a path that is substantially linear. Additionally, or alternatively, one or more of the friction elements 154 optionally extends along a path that is curved or arcuate.

For any of the embodiments, one or more of the friction elements 154 optionally comprises a plurality of teeth 160. The teeth 160 are similar to the teeth 60 of other embodiments described herein. The teeth 160 of one or more of the friction elements 154 are optionally arranged in a row that generally extends from proximate the anterior wall 142 to proximate the posterior wall 148. In embodiments, one or more of the friction elements 154 include teeth that are not arranged in a row.

Optionally, each friction element has from three to sixteen teeth 160. In some embodiments, one or more of the friction elements has eight teeth 160.

For any of the embodiments, the teeth 160 may optionally have a cross-sectional shape that is generally triangular. A first side 182 of a tooth extends away from an endplate 134. A second or long side 184 of the tooth extends from the endplate and intersects the first side 182 to define a free end 188 of the tooth. A fixed end 186 of the tooth engaged with the endplate is wider than the free end 188. In embodiments, the free end 188 defines a line, or is generally linear. Optionally, the free end is pointed.

In one embodiment, an edge of the first side 182 is oriented approximately parallel to a coronal plane 131. The coronal plane 131 is oriented substantially perpendicular to the median plane 132. Alternatively, the edge of the first side may be oriented at an oblique angle to the coronal plane 131.

An edge of the long side 184 is optionally oriented at an oblique angle to the coronal plane 131. In embodiments, the edge of the long side 184 is oriented at an angle of between about 35° and about 55°, or about 45° to the coronal plane 131.

The teeth have a predetermined height 190 defined by a height of the first side 182 relative to an endplate. In some embodiments, the height 190 is between about 0.5 mm and about 2.5 mm. In another embodiment, the height 190 is about 1.2 mm.

In embodiments, the first side 182 of one or more of the teeth is positioned toward the anterior wall 142 of the interbody device 130. Additionally, or alternatively, one or more of the teeth have a first side that is positioned toward the posterior wall 148 of the interbody device.

Notably, in some embodiments, the first sides 182 of each tooth define distinct planes. More specifically, any of the embodiments of the interbody device 130 may optionally include friction elements 154A, 154B, 154C, and 154D that are each oriented at a different angle 166 relative to the median plane 131 such that the first side 182 of a first tooth defines a first plane that is not coplanar with a first side of any other of the teeth 160. This may be seen by comparing FIGS. 16, 17 and 20. For example, FIG. 20 generally illustrates the second friction element 154B oriented such that the long sides 184 of its teeth are partially visible while only one edge of the first sides 182 of the teeth of friction element 154B are visible. In contrast, in FIG. 20 the third friction element 154C (illustrated below the second friction element 154B) is shown with the first sides 182 of its teeth visible. However, only a single edge of the long sides 184 of the teeth of the third friction element 154C are visible in FIG. 20.

Optionally, and referring again to FIGS. 16-17, the teeth 160 in a row of teeth may have free ends 188 that are spaced two or more distances from median plane 132. More specifically, a first tooth in friction element 154A may have a free end that is spaced a first distance laterally from the median plane. A second tooth in the friction element 154A may have a free end that is spaced a second distance laterally from the median plane. This arrangement of the teeth increases pull out resistance of the interbody device 130.

Optionally, for any of the embodiments, one or more of the teeth are rigid. Alternatively, in some embodiments, one or more of the teeth are flexible. The teeth can be integrally formed with the interbody device 130.

Referring again to FIG. 20, the anterior wall 142 has a height 143 measured relative to the coronal plane 131 that is greater than a height 149 of the posterior wall 148. In this manner, the shape of the interbody device 130 generally corresponds to the anterior and posterior walls of a disc to be replaced. Further, the larger height 143 of the anterior wall compared to the small height 149 of the posterior wall introduces a predetermined amount of curvature (or lordosis) between two adjacent vertebrae. In some embodiments, the height 143 of the anterior wall is between about 1.0 mm and about 2.0 mm, or about 1.4 mm greater than the height 149 of the posterior wall.

Because the anterior height 143 is greater than the posterior height 149, at least portions of the endplates 134A, 134B are oriented at an oblique angle relative to a transverse plane 133. The transverse plane 133 is substantially perpendicular to the median plane 132 and to the coronal plane 131.

In embodiments, a first plane 192A extending through superior ends of the anterior and posterior walls 142, 148 is oriented at an angle 194A of between about 2.0° and about 4.0°, or about 3.0° relative to the transverse plane 133. Additionally, or alternatively, a second plane 192B extending through inferior ends of the anterior and posterior walls 142, 148 is oriented at an angle 194B of between about −2.0° and about −4.0°, or about −3.0° relative to the transverse plane 133.

One or more of the endplates 134A, 134B may also be curved or arcuate between the anterior and posterior walls 142, 148 as generally illustrated in FIG. 20. In this manner, the endplates 134 may have a “domed” or convex shape configured to generally conform to a concave surface of a superior or inferior vertebrae. In contrast, some prior art cages have linear surfaces that do not conform to the surfaces of a vertebrae.

Because of the convex shape of the anterior and posterior walls 142, 148 according to some embodiments, a maximum height 196 of the interbody device 130 occurs between the anterior and posterior walls. In embodiments, the maximum height 196 is between 4 mm and 16 mm.

In embodiments, the position of the maximum height 196 is closer to the anterior wall than to the posterior wall. Accordingly, a first distance between the position of the maximum height 196 and the anterior wall 142 is less than a second distance between the position of the maximum height 196 and the posterior wall 148.

FIG. 20 also illustrates a maximum distance 198 between the teeth 160 of the interbody device 130. The distance 198 is measured approximately parallel to the coronal plane 131. In embodiments, the maximum distance 198 between teeth is between approximately 5 mm and 18 mm.

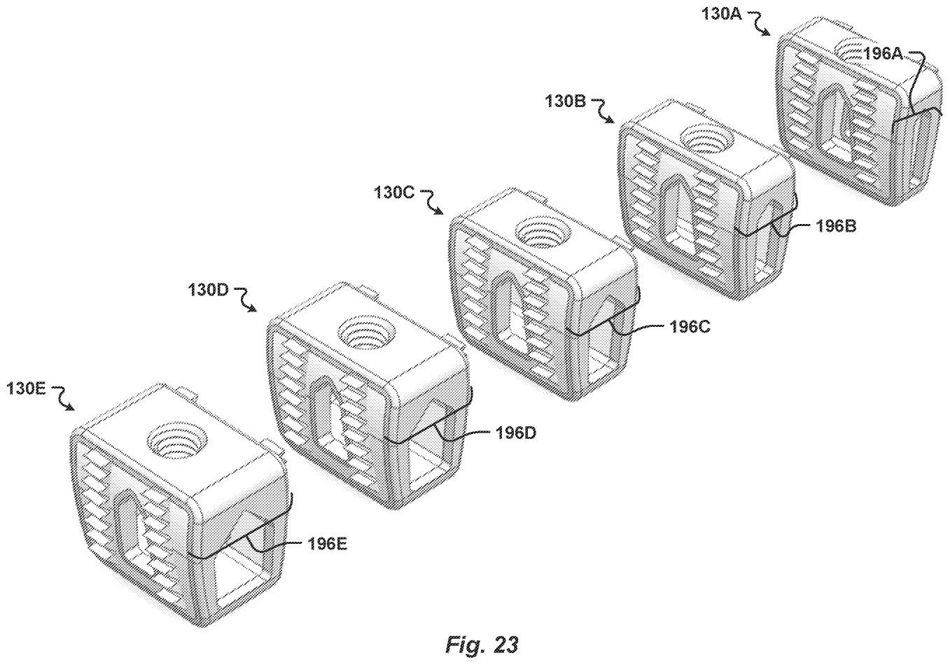

Referring now to FIG. 23, the interbody device 130 of the present disclosure may be formed with different maximum heights for use at different positions of a spine. Accordingly, a first interbody device 130A may have a maximum height 196A of between about 5.5 mm and about 6.0 mm. A maximum distance 198 between the teeth of the first interbody device is between 7.7 mm and 8.4 mm. The maximum height 196B of a second interbody device 130B is between about 6.5 mm and about 7.0 mm with a maximum distance 198 between the teeth of between 8.7 mm and 9.4 mm. A third interbody device 130C has a maximum height 196C of between about 7.5 mm and about 8.0 mm. The maximum distance 198 between the teeth of the third interbody device is between 9.7 mm and 10.4 mm. Another interbody device 130D has a maximum height 196D of between about 8.5 mm and about 9.0 mm and has a maximum distance 198 between its teeth of between 10.7 mm and 11.4 mm. The maximum height 196F of a fifth interbody device 130F is between about 9.5 mm and about 10.0 mm. The maximum distance 198 between the teeth of the fifth interbody device 130F is between 11.7 mm and 12.4 mm. Other sizes of the interbody devices 130 with different maximum heights 196 and maximum distances 198 are contemplated.

For any of the embodiments, the interbody device 130 may optionally be made of any known material or any material developed in the future known to those of skill in the art. In some embodiments, the interbody device comprises a plastic. The plastic may be a thermoplastic polymer. Optionally, the plastic is a polyether ether ketone (PEEK). Additionally, or alternatively, in other embodiments, the interbody device 130 comprises a metal. Optionally, for any of the embodiments, the metal is titanium or a titanium alloy. The titanium alloy may optionally be Ti-6Al-4V known to those of skill in the art.

In embodiments, the interbody device 130 is produced by an additive manufacturing process. In some embodiments, the interbody device is produced by a 3-D printing process.

The interbody device is optionally treated to produce micro- and nanoscale surface roughness. In embodiments, the treatment of the interbody device includes acid etching to promote the micro- and nanoscale surface roughness. Additionally, or alternatively, the interbody device is heat treated to promote the micro- and nanoscale surface roughness. Optionally, the exterior surfaces of the interbody device 130 are finished by shot blasting with titanium shot.

The interbody devices 30, 130 of the present disclosure provide many benefits compared to cages and disc replacement devices. For example, the interbody devices 30, 130 frictionally engage the vertebral bodies to resist pull out and inadvertent movement. Accordingly, the interbody devices of the present disclosure are retained in a disc space without the use of screws that extend through the interbody device into the vertebral bodies. Further, due to the frictional engagement of the interbody device with the vertebral bodies, a plate is not required to be fixed to the vertebral bodies to hold the interbody device in place.

By eliminating the use of screws and plates, the interbody devices of the present disclosure reduce presurgical planning and materials used to perform a spinal fusion. Further, surgical time is reduced because the interbody devices can be positioned in the disc space more quickly. The surgical procedure is also simplified by eliminating the need for anchoring devices such as screws, rods, pins or anchors that must penetrate one or more vertebral body. Specifically, damage to vertebral bodies is reduced by eliminating the use of screws, rods, pins, deployable anchors and plates. Eliminating the use of secondary anchors, such as screws, pins, plates, and rods also reduces the number of instruments used during spinal fusion which decreases the risk of injury to critical structures in the patient' neck or abdomen.

In some embodiments, the interbody devices 30, 130 of the present disclosure are static and do not include deployable or moveable anchors, pins, shims, or screws. In addition to reducing the number of instruments and tools used during surgery and reducing or eliminating the need to cut or penetrate a vertebral body, the static interbody devices also reduce the risk of failure of the interbody device. For example, in some prior art cages have moving parts that may inadvertently become loose or unlock and unintentionally allow the cage to move. Additionally, deployable fixation devices (such as screws, pins and anchors) may bend, shear, or break under anatomical loads. This can lead to migration of loose pieces of the fixation device in the patient's body. This requires revisional surgery to remove pieces of the fixation device as well as additional surgery to correct for potential pseudo arthrosis. In contrast, because there are no moving parts in the static interbody devices 30, 130 of embodiments of the present disclosure, there are no moving parts that can become loose, unlock, break, or otherwise fail.

The interbody devices will allow for fusion from one vertebral body to another without the use of either autologous or donor bone reducing the cost and/or risk of the surgery. The number of components of the interbody device is greatly reduced compared to cages allowing for further reduction of costs. Lastly, without the screws, deployable anchors or alternative fixation mechanism, the overall design can be more highly optimized to fit within the interbody space.

While various embodiments of the system have been described in detail, it is apparent that modifications and alterations of those embodiments will occur to those skilled in the art. It is to be expressly understood that such modifications and alterations are within the scope and spirit of the present disclosure. Features of one aspect or embodiment of the present disclosure may be combined with other aspects or embodiments disclosed herein. It is contemplated that various aspects, features and devices shown and/or described with respect to one embodiment may be combined with (or substituted for) aspects, features or devices of other embodiments regardless of whether or not such a combination or substitution is specifically shown or described herein. For example, the position, arrangement, and/or alignment of friction elements 54, 154 of embodiments of the present disclosure may be combined with any interbody device 30, 130 described herein. Additionally, or alternatively, a keel 56, projection 58 and teeth 60, 160 can be used with any embodiment of the interbody devices 30, 130 of the present disclosure.

Further, it is to be understood that the phraseology and terminology used herein is for the purposes of description and should not be regarded as limiting. The use of “including,” “comprising,” or “having” and variations thereof herein are meant to encompass the items listed thereafter and equivalents thereof, as well as, additional items.

To provide additional background, context, and to further satisfy the written description requirements of 35 U.S.C. § 112, the following references are incorporated by reference herein in their entireties: CONDUIT™ Interbody Platform EIT™ Cellular Titanium, Sales Sheet, available at https://www.jnjmedicaldevices.com/sites/default/files/user uploaded assets/pdf_assets/2019-05/Conduit%20Interbody-%20EIT%20Sales%20Sheet.pdf; U.S. Pat. Nos. 8,034,110; 8,083,799; 8,460,385; 10,098,755; 10,744,003; U.S. Pat. Pub. 2008/0269901; U.S. Pat. Pub. 2012/0277870; U.S. Pat. Pub. 2015/0305887; U.S. Pat. Pub. 2019/0105174; and PCT Pub. WO 2017/205623.

### Example

Samples of three interbody devices 130 of the present disclosure were evaluated using a “static expulsion” method to determine the loads required to expulse or displace the interbody device 130 from simulated bone of varied densities. The samples were of the “6 mm” embodiment of the interbody device 130B generally illustrated in FIG. 23. Each interbody device was tested three times using simulated bone of three different densities: Grade 5 (least dense), Grade 15 (medium density) and Grade 40 (most dense).

For comparison, the same static expulsion tests were performed using three samples of the AIS-C Cervical Stand-Alone System produced by Genesys Spine of Austin, Tex. The Genesys Spine AIS-C Cervical Stand-Alone System includes PEEK interbodies and titanium interbodies, which utilize an integrated titanium alloy locking mechanism. Both PEEK interbodies and titanium interbodies of the Genesys device are configured to be anchored to patient anatomy by two titanium alloy bone anchors. The titanium alloy cervical bone anchors provide integrated fixation for the system and are to be offered in various lengths. Notably, the bone anchors are movable and are deployed through channels in the interbody and embed into the adjacent cervical vertebrae.

This testing indicates that the interbody device 130 of the present disclosure performs similarly to the Genesys Spine AIS-C Cervical Stand-Alone System which has been approved for use by the U.S. Food and Drug Administration. Specifically, these results suggest that the interbody device 130 can resist pushout forces and the anticipated physiologic loads expected to be experienced after insertion in a disc space. Further, the testing shows that the interbody device 130 is operable to frictionally engage simulated bone using only static friction elements and withstand expulsion in a manner similar to the Genesys Spine AIS-C Cervical Stand-Alone System which includes deployable bone anchors.

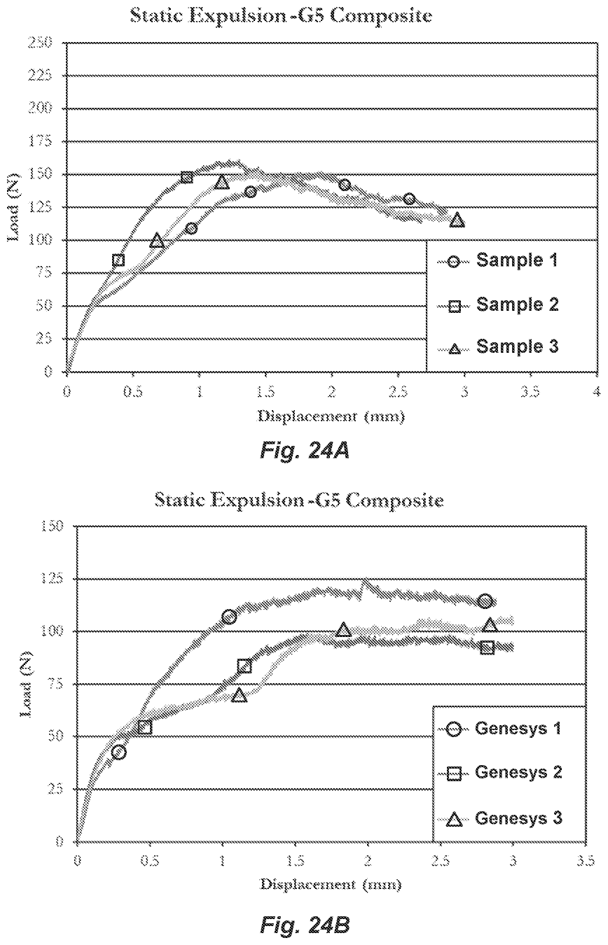

FIG. 24A is a graph of the performance of the three samples of the interbody devices 130 in the static expulsion test conducted with simulated bone of Grade 5. Table 1, below, illustrates the results of the static expulsion test for the interbody devices 130 conducted with simulated bone of Grade 5.

TABLE 1

| | | Displacement |
|---|---|---|
| | Peak Load | at Peak Load |
| Specimen | (N) | (mm) |
| Sample 1 | 152 | 1.91 |
| Sample 2 | 161 | 1.23 |
| Sample 3 | 153 | 1.44 |
| Mean | 155 | 1.53 |
| Standard Deviation | 5.1 | 0.348 |

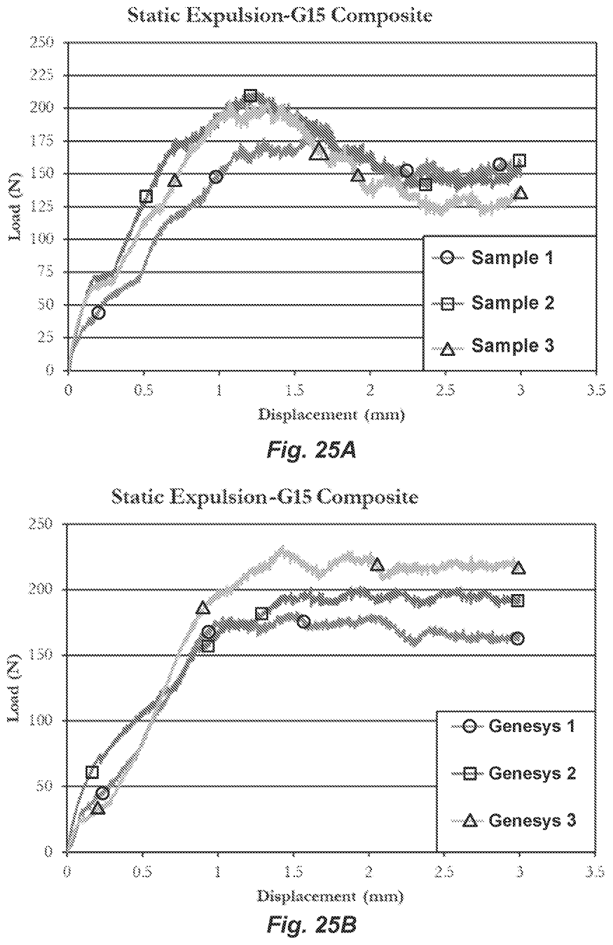

FIG. 25A is a graph of the performance of the three samples of the interbody devices 130 in the static expulsion test conducted with simulated bone of Grade 15. Table 2 illustrates the results of the static expulsion test for the interbody devices 130 conducted with simulated bone of Grade 15.

TABLE 2

| | | Displacement |
|---|---|---|
| | Peak Load | at Peak Load |
| Specimen | (N) | (mm) |
| Sample 1 | 189 | 1.67 |
| Sample 2 | 212 | 1.25 |
| Sample 3 | 206 | 1.07 |
| Mean | 202 | 1.33 |
| Standard Deviation | 11.9 | 0.308 |

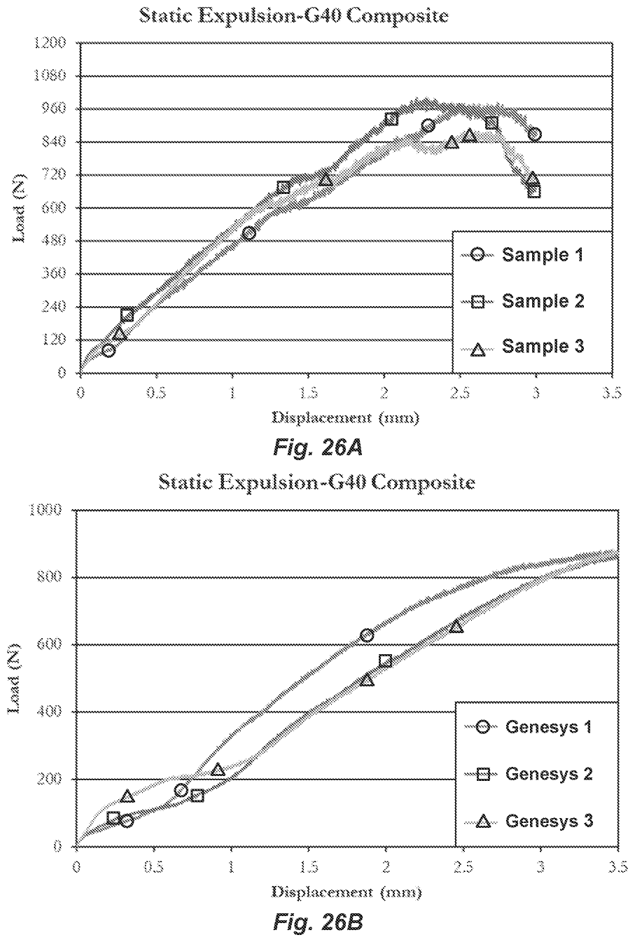

FIG. 26A is a graph of the performance of the three samples of the interbody devices 130 in the static expulsion test conducted with simulated bone of Grade 40. Table 3 illustrates the results of the static expulsion test for the interbody devices 130 conducted with simulated bone of Grade 40.

TABLE 3

| | | Displacement |
|---|---|---|
| | Peak Load | at Peak Load |
| Specimen | (N) | (mm) |
| Sample 1 | 982 | 2.75 |
| Sample 2 | 1,000 | 2.21 |
| Sample 3 | 880 | 2.60 |
| Mean | 954 | 2.52 |
| Standard Deviation | 64.7 | 0.279 |

For comparison, FIG. 24B is a graph of the performance of the three samples of the Genesys Spine AIS-C Cervical Stand-Alone System with simulated bone of Grade 5. Table 4 illustrates the results of the static expulsion test for the samples of the Genesys Spine AIS-C Cervical Stand-Alone System with simulated bone of Grade 5.

TABLE 4

| | | Displacement |
|---|---|---|
| | Peak Load | at Peak Load |
| Specimen | (N) | (mm) |
| Genesys sample 1 | 125 | 1.99 |
| Genesys sample 2 | 99 | 1.58 |
| Genesys sample 3 | 107 | 2.99 |
| Mean | 110 | 2.19 |
| Standard Deviation | 13.3 | 0.725 |

FIG. 25B is a graph of the performance of the three samples of the Genesys Spine AIS-C Cervical Stand-Alone System with simulated bone of Grade 15. Table 5 illustrates the results of the static expulsion test for the samples of the Genesys Spine AIS-C Cervical Stand-Alone System with simulated bone of Grade 15.

TABLE 5

| | | Displacement |
|---|---|---|
| | Peak Load | at Peak Load |
| Specimen | (N) | (mm) |
| Genesys sample 1 | 183 | 1.52 |
| Genesys sample 2 | 202 | 1.94 |
| Genesys sample 3 | 234 | 1.43 |
| Mean | 206 | 1.63 |
| Standard Deviation | 25.8 | 0.272 |

FIG. 26B is a graph of the performance of the three samples of the Genesys Spine AIS-C Cervical Stand-Alone System with simulated bone of Grade 40. Table 6 illustrates the results of the static expulsion test for the samples of the Genesys Spine AIS-C Cervical Stand-Alone System with simulated bone of 40.

TABLE 6

| | | Displacement |
|---|---|---|
| | Peak Load | at Peak Load |
| Specimen | (N) | (mm) |
| Genesys sample 1 | 882 | 3.46 |
| Genesys sample 2 | 867 | 3.50 |
| Genesys sample 3 | 881 | 3.47 |
| Mean | 877 | 3.47 |
| Standard Deviation | 8.4 | 0.021 |

1. An interbody device configured for use in a spinal fusion procedure to frictionally engage an upper vertebral body and a lower vertebral body, comprising:
a superior endplate opposite to an inferior endplate;
a first superior friction element extending from the superior endplate, wherein the first superior friction element is oriented at an oblique angle to a median plane that extends through the superior and inferior endplates and bisects the interbody device;
a first inferior friction element extending from the inferior endplate; and
an anterior wall including an engagement feature.

2. The interbody device of claim 1, further comprising a second superior friction element extending from the superior endplate, the first superior friction element being on a first side of the median plane and the second superior friction element being on a second side of the median plane.

3. The interbody device of claim 2, wherein the second superior friction element is oriented at an oblique angle to the median plane and to the first superior friction element.

4. The interbody device of claim 3, wherein the first superior friction element is oriented at a first angle relative to the median plane and the second superior friction element is oriented at a second angle relative to the median plane.

5. The interbody device of claim 4, wherein the second angle is a positive angle, and wherein the first angle is a negative angle that is of approximately equal magnitude to the second angle.

6. The interbody device of claim 4, further comprising a second inferior friction element extending from the inferior endplate, the second inferior friction element being on the first side of the median plane and the first inferior friction element being on the second side of the median plane.

7. The interbody device of claim 6, wherein the first inferior friction element is oriented at a third angle relative to the median plane that is approximately equal to the first angle such that the first inferior friction element is approximately parallel to the first superior friction element, and wherein the second inferior friction element is oriented at a fourth angle relative to the median plane that is approximately equal to the second angle such that the second inferior friction element is approximately parallel to the second superior friction element.

8. The interbody device of claim 1, wherein the friction elements each comprise a plurality of teeth arranged in a row.

9. The interbody device of claim 8, wherein the teeth include:
a first side extending away from one of the superior and inferior endplates, the first side being proximate to the anterior wall;
a long side that is oriented at an oblique angle to the first side; and
a free end defined by an intersection of the long side with the first side.

10. The interbody device of claim 9, wherein the first side is approximately planar and the long side is approximately planar.

11. The interbody device of claim 1, further comprising a posterior wall positioned opposite to the anterior wall, wherein the posterior wall has a first height that is less than a second height of the anterior wall.

12. The interbody device of claim 11, wherein the superior endplate extends along a curved path between the anterior wall and the posterior wall such that the superior endplate is convex between the anterior and superior walls.

13. The interbody device of claim 1, wherein one or more of the superior endplate and the inferior endplate comprise a micro porous material for bone ingrowth.

14. The interbody device of claim 1, wherein the interbody device comprises a PEEK.

15. The interbody device of claim 1, wherein the interbody device comprises a metal.

16. The interbody device of claim 1, wherein the interbody device is rigid.

17. The interbody device of claim 1, wherein the interbody device is produced by an additive manufacturing process.

18. The interbody device of claim 1, wherein the interbody device is configured to frictionally engage the upper vertebral body and the lower vertebral body without the use of a screw and without the use of a plate.

19.-26. (canceled)

27. A method of forming an interbody device configured for use in a spinal fusion procedure which is configured to engage one or more of an upper vertebral body and a lower vertebral body, comprising:
forming the interbody device with a superior endplate opposite to an inferior endplate;
forming a first friction element extending from the superior endplate, the first friction element extending at a first oblique angle to a median plane extending through the endplates and bisecting the interbody device;
forming a second friction element extending from the superior endplate, the second friction element extending at a second oblique angle to the median plane;
forming a third friction element extending from the inferior endplate, the third friction element extending approximately parallel to the first friction element;
forming a fourth friction element extending from the inferior endplate, the fourth friction element extending approximately parallel to the second friction element;
forming an anterior wall extending between the superior and inferior endplates; and
forming an engagement feature that includes an aperture extending through the anterior wall to an interior cavity within the interbody device.

28. The method of claim 27, wherein the friction elements each comprise a plurality of teeth arranged in a row.

29. The method of claim 27, wherein the interbody device is produced by an additive manufacturing process.

30. The method of claim 27, wherein the interbody device is static and is formed of one or more of a PEEK and a metal.
